(19) United States
(12) Patent Application Publication
(10) Pub. No.: US 2021/0008251 A1
(43) Pub. Date: Jan. 14, 2021

(54) Fractionating Extracellular Matrix to Modulate Bioactivity and the Host Response

(71) Applicant: University of Pittsburgh - Of the Commonwealth System of Higher Education, Pittsburgh, PA (US)

(72) Inventors: Stephen F. Badylak, West Lafayette, IN (US); Peter F. Slivka, Westborough, MA (US)

(21) Appl. No.: 16/844,382

(22) Filed: Apr. 9, 2020

Related U.S. Application Data
(63) Continuation of application No. 15/306,262, filed on Oct. 24, 2016, now abandoned.
(60) Provisional application No. 61/983,507, filed on Apr. 24, 2014.

(51) Int. Cl. A61L 27/36; A61K 35/12; A61K 9/00; A61K 35/22; A61L 27/52

(52) U.S. Cl. CPC A61L 27/3633; A61K 35/12; A61K 9/0014; A61K 35/22; A61L 2430/40; A61L 27/3691; A61L 27/52; A61L 2300/606; A61L 2430/30; A61L 27/3687

(57) ABSTRACT

Provided herein are methods of fractionating extracellular matrix (ECM) materials, producing soluble and structural fractions having different immunological activities. Also provided are compositions and devices comprising the fractions. A method of immune modulation also is provided in which an amount of a soluble or structural ECM fraction prepared according to the methods provided herein is administered to a patient in an amount effective to modulate immune function, for example macrophage function.

### CROSS REFERENCE TO RELATED APPLICATIONS

This application is a continuation of U.S. patent application Ser. No. 15/306,262, filed Oct. 24, 2016, which is a national stage of International Patent Application No. PCT/US2015/027498, filed Apr. 24, 2015, which claims the benefit of U.S. Provisional Patent Application No. 61/983,507, filed Apr. 24, 2014, each of which is incorporated herein by reference in its entirety.

Mammalian ECM derived from a variety of tissue sources has been extensively utilized as a surgical mesh material for many clinical applications including hernia repair, rotator cuff repair, breast reconstruction, and musculotendinous reinforcement. When properly prepared and implanted these 2-D sheets of ECM have been shown to function as an inductive template for the repair and regeneration of damaged or missing tissues. In vivo preclinical studies have established a temporal sequence for ECM mediated constructive remodeling beginning with a transient leukocyte response followed by a dense mononuclear cell infiltration into the ECM scaffold. Subsequently these invading cells degrade the scaffold and deposit new ECM as well as small pockets of site appropriate, vascularized, and innervated tissue.

The pathophysiology of ECM scaffold remodeling has been partially characterized and includes the recruitment of endogenous stem/progenitor cells as well as modulation of the innate immune response towards a regulatory (M2/Th2) phenotype. Macrophages infiltrating ECM scaffolds have been shown to express a number of M2 markers including CD206 and CD163. Moreover, the presence of these M2 markers appears to be a strong predictor of a constructive host remodeling response.

While 2-D sheets of ECM have shown clinical utility, the lack of a third dimension and constraint to a sheet form has limited the potential applications for these materials. More recently, ECM scaffolds have been processed into different forms including powders, putties, and hydrogels. Of these alternative forms, hydrogels present the most options as they can be cast into 3-D shapes for cell culture and tissue engineering applications or injected directly into host tissue. These injected hydrogels can fill complex cavities and retain the shape of that cavity once polymerized. Generation of hydrogels from ECM requires ECM to be solubilized, which is readily accomplished with pepsin digestion. Pepsin digestion has been extensively utilized to prepare collagen both for research and medical purposes and is an established industry standard. Importantly, pepsin solubilized ECM hydrogels have been shown to elicit a similar host response to the 2-D surgical mesh form of ECM.

### SUMMARY

Degradation products of extracellular matrix (ECM) have been widely shown to improve tissue remodeling outcomes when placed at a site of injury. Hydrogels of ECM which concentrate these degradation products have produced similar results. Isolated molecules of ECM (i.e. collagen, fibronectin, cecropins, etc.) have been utilized to mimic a specific biological activity of ECM (i.e. cell adhesion, antimicrobial activity, etc.). However, these isolated components often fail to, or only partially, recapitulate the bioactivity of ECM. The present invention describes a method for fractionating extracellular matrix (ECM) by separating the soluble and structural components of the material. Separating the components alters the bioactivity the two fractions with certain aspects of the bioactivity being enhanced several fold and others almost completely diminished. Enriching the activity of the fractions in this way preserves the molecular complexity of the material while providing a methodology to tailor the material to elicit specific cellular and host responses. Tailoring the bioactivity in this manner could improve the overall host response to the materials in vivo. Additionally, the tailor made activity could be utilized to drive cellular responses in vitro as an additive to culture media.

Current technology uses single isolated components of ECM. The current invention takes advantage of the cadre of bioactive molecules in ECM and simply enhances the activity of those molecules by limited fractionation.

A method of preparing one or more biologically active fractions of ECM is provided. The method if useful for modulating chemotaxis and proliferation of stem cells, and for modulating an immune response. The method comprising: partially or completely digesting with an acid protease, such as pepsin, decellularized ECM material prepared from a tissue; neutralizing the digested ECM material to a pH of 7.0-8.0, 7.2-7.8 or 7.4; gelling the neutralized, digested ECM material at a temperature above its Lower Critical Solution Temperature; centrifuging the gelled ECM material to produce a pellet and a supernatant; and separating the supernatant and the pellet thereby separating a structural and a soluble fraction of the ECM material.

According to one embodiment, the decellularized ECM material prepared from the tissue is not dialyzed prior to the partial or complete digestion with the acid protease and/or is not dialyzed after digesting with an acid protease and before gelling of the neutralized, digested ECM material. In one embodiment, the method further comprising dispersing the pellet/structural fraction into an aqueous solution, such as water, saline, isotonic buffer, PBS, or serum-free medium, thereby preparing a solution of structural components of the ECM. Pellet dispersal can be accomplished, for example, by homogenization, for example, in an aqueous solution.

In another embodiment, the supernatant/soluble fraction is further purified by precipitating remaining structural components from the supernatant, for example, by salting out those structural components—that is by increasing the salt concentration in the supernatant. Either or both of the structural fraction and the soluble fraction are optionally dried, for example by lyophilization and then might be re-hydrated using an appropriate aqueous solution, such as water, saline, isotonic buffer, PBS, or serum-free medium. According to one embodiment, the supernatant is concentrated. That is, the lyophilized supernatant is re-hydrated to a volume, less than the volume of the supernatant before lyophilization, optionally the lyophilized supernatant is re-hydrated to a volume <10%, 10%, 20%, 25% or 50% of the volume of the supernatant before lyophilization, thereby producing a concentrated solution of soluble ECM components.

In one embodiment, the decellularized ECM material is partially digested by the acid protease. In one example, the decellularized ECM material is digested less completely than a digestion of 1 mg/mL lyophilized, powdered ECM material with 1 mg/mL pepsin in 0.01 M HCl for 48 hours. In another example, the decellularized ECM material is digested less completely than a digestion of 10 mg/mL lyophilized, powdered ECM material with 1 mg/mL pepsin in 0.01 M HCl for 48 hours. In one further embodiment, hyaluronic acid in the ECM material is digested less than 50%, 40%, 30%, 25%, 20% or 10% as compared to undigested ECM material.

According to one embodiment, the biologically active fraction of ECM composition prepared by any method described herein is absorbed into, adsorbed onto, or otherwise dispersed onto or into a biocompatible substrate. Non-limiting examples of a biocompatible substrate include: a mesh, a non-woven, decellularized tissue, a polymer composition, a polymeric structure, a cell growth scaffold, an implant, an orthopedic implant, and intraocular lens, sutures, intravascular implants, stents, and transplants.

A biologically active fraction of ECM-containing composition also is provided, that is prepared by any method of preparing a biologically active fraction of ECM described herein, such as by: partially or completely digesting with an acid protease, such as pepsin, decellularized ECM material prepared from a tissue; neutralizing the digested ECM material to a pH of 7.0-8.0, 7.2-7.8 or 7.4; gelling the neutralized, digested ECM material at a temperature above its Lower Critical Solution Temperature; centrifuging the gelled ECM material to produce a pellet and a supernatant; and separating the supernatant and the pellet thereby separating a structural and a soluble fraction of the ECM material.

A for supporting tissue remodeling, cell growth, migration and/or differentiation, also is provided. The device comprises a comprising a biocompatible substrate, such as, without limitation, a gel, mesh, polymer and/or ECM-containing material, and a biologically active fraction of ECM-containing composition, that is prepared by any method of preparing a biologically active fraction of ECM described herein, such as by: partially or completely digesting with an acid protease, such as pepsin, decellularized ECM material prepared from a tissue; neutralizing the digested ECM material to a pH of 7.0-8.0, 7.2-7.8 or 7.4; gelling the neutralized, digested ECM material at a temperature above its Lower Critical Solution Temperature; centrifuging the gelled ECM material to produce a pellet and a supernatant; and separating the supernatant and the pellet thereby separating a structural and a soluble fraction of the ECM material, wherein the composition absorbed into, adsorbed onto, or otherwise dispersed on or in the biocompatible substrate.

According to a further embodiment, a method of modulating an immune response in a patient in need thereof is provided. The method comprises comprising administering to a patient parenterally or topically the biologically active fraction of ECM according to any embodiment described herein, in an amount effective to modify an immune response in the patient. The method comprises, for example, administering the soluble ECM components from the supernatant to the patient, thereby increasing the macrophage M2 response in the patient. Alternatively, the method comprises administering the structural ECM components from the supernatant to the patient, thereby increasing the macrophage M1 response in the patient.

### BRIEF DESCRIPTION OF THE DRAWINGS

FIG. 1. Fractionation of UBM into Soluble and Structural Fractions. A, Fractions were generated by forming a UBM hydrogel and centrifuging the structural components into a pellet. The liquid supernatant was removed, dried, and finally rehydrated in 10% of the original volume to increase the salt concentration and salt out any remaining structural components. Both the structural and soluble components isolated in this procedure were diluted to their original volume to allow for direct comparison of their activity. B, Representative SDS PAGE gel with equal protein loading for UBM Digest, Structural Components and Soluble Components. C, Low molecular weight bands are shown with 3 times additional protein loading. D, Picrosirius red stain of the structural components confirms the presence of collagen.

FIG. 2. Chemotaxis of Perivascular Stem Cells Towards a UBM Hydrogel and its Soluble and Structural Components. A, PSCs were serum starved overnight, loaded into Boyden chemotaxis chambers, and allowed to migrate for 3 hours through an 8 μM collagen-coated, polycarbonate filter towards the materials shown above. Data are expressed as a fold-increase in the number of chemotaxing cells compared to a digestion enzyme only control (163+35 cells), which was normalized to zero. Error bars indicate the standard error in the measurement for three experiments with four replicates per experiment. No significant differences were found between different materials at a given concentration. B, Representative 10× magnification mosaic images from one well of migrated cells for each material at the highest concentration tested as well as the digestion enzyme only control are shown. Nuclei are shown in white. More nuclei indicate a stronger recruitment effect.

FIG. 3. Changes in Proliferation of Perivascular Stem Cells After Exposure to UBM Hydrogel and its Soluble and Structural Components. A, PSCs were exposed to UBM or its fractionated components along with BrdU to measure changes in proliferation. The number of co-labeling (DAPI⁺BrdU⁺) nuclei was determined and the fold changes co-labeling nuclei against a digestion enzyme only control (52+4, normalized to zero) are shown. Error bars indicate the standard error in the measurement for three unique experiments with three replicates per experiment. No significant differences were found between materials at any of the concentrations tested. B, Representative 10× magnification images of BrdU+ nuclei from PSCs treated with UBM hydrogel, its components, or digestion enzyme only control at the highest concentration tested for each condition. Greater numbers of DAPI+ nuclei indicate more proliferation.

FIG. 4. Macrophage Phagocytosis of Latex Beads in Response to UBM and its Fractionated Components. A, THP-1 human monocytes were differentiated to macrophages with PMA and rested. Macrophages were treated with the specified conditions for 48 hours and exposed to latex particles. The percentage of phagocytosing cells determined by flow cytometry are shown. Error bars indicate the standard deviation for six unique replicates. Statistically significant increases in phagocytosis are shown for the soluble components (#) and digestion enzyme control (*). B, Representative dot plots of macrophages phagocytosing latex beads after treatment with UBM hydrogel, soluble, or structural components and digestion enzyme only control.

FIGS. 5A-5B. TNFα and IL-1β Secretion After Treatment with UBM and its Fractionated Components. A,B, THP-1 human monocytes were differentiated to macrophages with PMA, rested, and treated with the specified conditions for 48 hours. After treatment the concentration of TNFα and IL-1β in culture supernatants was determined using commercially available ELISA kits. C,D, Macrophages treated in the same way as A and B were challenged with LPS (100 ng/mL) to determine if treatment with the materials could prevent inflammation. The concentration of TNFα and IL-1β in culture supernatants was once again determined using commercially available ELISA kits. Significant differences between groups are denoted with symbols (*, #, ¥). The data presented here represent a total of three replicates with each replicate representing duplicate samples. None of the components significantly increased TNFα secretion. The structural components increased IL-1β secretion above control but minimally compared to LPS. Both UBM digest and the soluble components significantly prevented TNFα secretion. Only the soluble components significantly prevented IL-1β secretion.

FIG. 6. Effect of UBM and its Fractionated Components on IL1-RA Secretion. THP-1 human monocytes were differentiated to macrophages with PMA, rested and treated with UBM, soluble, or structural components for 48 hours. IL-1RA in culture supernatants was measured using a commercially available ELISA kit. The data presented here represent a total of three replicates with each replicate representing duplicate samples. Significant differences between groups are denoted with symbols (*, #). All three materials significantly increased IL1-RA secretion above control while the soluble components were also significantly stronger than UBM hydrogel and the structural components.

FIG. 7. PGE2 and PGF2a Secretion After Treatment with UBM and its Fractionated Components. THP-1 human monocytes were differentiated to macrophages with PMA, rested, and treated with the specified conditions for 72 hours. The concentrations of PGE2 (A) and PGF2α (B) in culture supernatants were determined using commercially available ELISA kits. The requirement of COX2 in prostaglandin production was investigated by coadministering the COX2 inhibitor, NS-398 along with UBM hydrogel (C). Significant differences between groups are denoted with an asterisk (*). The data presented here represent a total of three replicates with each replicate representing duplicate samples. Both UBM digest and the structural components significantly increased the concentrations of PGE2 and PGF2α above no treatment and digestion enzyme only control, while the soluble components did not.

FIG. 8. Evaluation of COX2 Expression by Invading Macrophage In Vivo. Sprague-Dawley rats had a 1×1 cm square portion of the internal and external oblique removed from the abdominal wall into which a 1×1×0.5 cm UBM hydrogel was placed. The animals were sutured closed and survived for 3 and 7 days. A, Histological sections were co-immunolabeled for CD68 (Alexa 594) and COX2 (Alexa 488) expression. Nuclei were stained with DAPI. Representative 32× images for both timepoints are depicted. B, Explants were also co-immunolabeled for CD206 (Alexa 594) and COX2 (Alexa 488) expression. At both timepoints, COX2 was found to colocalize with both CD68 and CD206.

FIG. 9. Minimal digestion of structural components increases bioactivity of UBM digest. UBM digests were prepared where the pepsin concentration was reduced from 1 mg/mL in 10 fold serial dilutions. THP1 cells were incubated with UBM digests at a final concentration of 1 mg/mL for 48 hours. Prostaglandin E2 (PGE2) production from these cells is shown.

FIG. 10. Treatment of primary rat bone marrow derived macrophages with UBM digest increases PGE2 production. However, UBM digest that has been additionally treated with hyaluronidase (HA) to degrade the structural component hyaluronan, does not cause an increase in macrophage PGE2 production.

FIG. 11. Confirmation of Aspirin Administration. (A) The systemic concentration of salicylates was measured in the blood of animals after 7 days of aspirin treatment. Concentrations were determined using a commercially available ELISA kit. Significant differences (p<0.05) in salicylate content are shown.

FIG. 12. The effect of aspirin administration on UBM stimulated collagen deposition in vivo. Tissue sections from the specified time points were stained with picrosirius red and imaged using polarized light microscopy. (A) Representative images of tissue sections from untreated and aspirin treated animals 35 days post operatively are shown. The color hue of the fibers represents the relative collagen thicknesses (in order of thinnest to thickest): green, yellow, orange, and red in original and shades of gray as indicated in (B). (B) The total area and proportion of collagen thickness in non-treated (NT) and Aspirin treated (ASPIRIN) animals after 3, 7, 14, and 35 days was assessed utilizing an automated MatLab script. Significant decreases in total collagen deposition are shown (p<0.05).

FIG. 13. The effect of aspirin administration on UBM stimulated myogenesis in vivo. Tissue sections from the specified time points were MHC stained and imaged. (A) Representative images of tissue sections from untreated and aspirin treated animals 35 days post operatively are shown. (B) The myogenic index (total cross sectional area of MHC+ cells within the defect expressed as a function of the total defect area) was quantified at 35 days for untreated and aspirin treated animals. Significant decreases in myogenesis are shown (p<0.05).

FIG. 14. The effect of aspirin administration on overall macrophage phenotype in vivo during UBM mediated constructive remodeling. Tissue sections from the specified time points were immunolabeled for CD68 (pan-macrophage), CD206 (M2), and CD86 (M1) and imaged. Four representative images of each tissue section were collected and the number of CD68+CD206+ and CD68+CD86+ cells were quantified using Cell Profiler. (A) Representative images of tissue sections from untreated and aspirin treated animals are shown. (B) The average M2/M1 ratio in the tissue sections is expressed as a ratio of CD68+CD206+ cells to CD68+CD86+ cells. A statistically significant main effect (averaged data over all time points) for the untreated vs. aspirin treated groups is shown (p<0.05).

FIG. 15. The effect of aspirin administration on CD206 and CD86 expression in macrophages in vivo. Tissue sections from the specified time points were immunolabeled for CD68 (pan-macrophage), CD206 and CD86. Four representative images of each tissue section were collected and the number of CD68+CD206+ cells (A) and CD68+CD86+ cells (B) per field of view was quantified using Cell Profiler. NSAID administration resulted in an overall decrease and increase in M2 and M1 cells, respectively. A statistically significant (p<0.05) main effect was observed for both CD206+ and CD86+.

FIG. 16. The effect of UBM on COX2 expression and PGE2 secretion in THP1 cells. THP1 monocytes were differentiated to a macrophage-like cell lineage with PMA for 24 hours and rested for an additional 24 hours. After which, cells were stimulated with UBM hydrogel (0.5 mg/mL). Cell lysates were collected at 4, 8, and 24 hours, resolved on SDS PAGE gels, and immunoblotted for COX2 expression. Untreated cells collected at the 0 hour time point served as an expression control and actin was utilized as a loading control. (A) Representative immunoblots for aspirin treated and untreated cells are shown. (B) Relative changes in COX2 expression were determined using densitometry. The average change in COX2 expression is shown. Significant differences in COX2 expression (p<0.05) are shown.

FIG. 17. The effect of Aspirin on PGE2, PGF2a, and IL-1RA production in UBM hydrogel treated macrophages in vitro. THP1 monocytes were differentiated to a macrophage-like cell lineage with PMA for 24 hours and rested for an additional 24 hours. Aspirin (200 μM) was added to cells for 1 hour prior to UBM hydrogel (0.5 mg/mL) addition. Cells were incubated for 48 hours and culture supernatants were collected. (A) PGE2, (B) PGF2a, and (C) IL-1RA concentrations in the culture supernatants were determined using commercially available ELISA kits. Significant differences in secreted factor concentration are shown (p<0.05).

FIG. 18. The effect of UBM hydrogel on TNFα, IL-1β, and PGE2 secretion in macrophages in vitro. THP1 monocytes were differentiated to a macrophage-like cell lineage with PMA for 24 hours and rested for an additional 24 hours. UBM hydrogel (0.5 mg/mL) was added and cells were incubated for 24, 48, or 72 hours. After incubation culture supernatants were collected and secreted factor concentrations were determined using commercially available ELISA kits. Significant differences in secreted factor concentration are shown (p<0.05).

FIG. 19. PGE2 secretion and inhibition in THP1, primary BMDM, and primary microglia with small molecule inhibitors. THP1 monocytes were differentiated to a macrophage-like cell lineage with PMA for 24 hours and rested for an additional 24 hours. BMDMs and microglia were prepared as described and seeded in 96 well plates. After seeding, cells were pre-treated with inhibitors for 1 hour then stimulated with UBM hydrogel (0.5 mg/mL) for 48 hours. Culture supernatants were collected and PGE2 levels were quantified using commercially available ELISA kits. NSAID mediated reductions in absolute quantities of PGE2 for THP1, BMDM, and microglia are shown. Significant differences in PGE2 secretion are shown.

FIG. 20. The effect of aspirin treatment on CD206 and CD86 expression in macrophages treated with UBM hydrogel in vitro. THP1 monocytes were differentiated to a macrophage-like cell lineage with PMA for 24 hours and rested for an additional 24 hours. Aspirin (200 μM) was added to cells for 1 hour prior to UBM hydrogel (0.5 mg/mL) addition. Cell lysates were collected at 4, 8, and 24 hours, resolved on SDS gels, transferred to membranes, and immunoblotted for CD206 and CD86 expression. Actin served as a loading control. (A) Representative blot of CD206 expression and (B) relative changes in CD206 expression compared to 0 hr control measured with densitometry. (C) Representative blot of CD86 expression and (D) relative changes in CD86 expression compared to 0 hr control measured with densitometry. Significant differences in CD206 and CD86 expression between aspirin treated and untreated cells are shown (p<0.05).

FIG. 21. The effect of aspirin treatment on myotube formation in UBM hydrogel treated co culture system. THP1 monocytes were differentiated with PMA and rested in a transwell insert. (A) Representative images of C2C12 myoblast fusion into MHC+ myotubes (bright) stimulated with UBM and inhibited with Aspirin. (B) Quantitative image analysis of several indices of myogenesis. All data are expressed as a percentage change from digestion enzyme only control.

FIG. 22 shows schematically one embodiment of a femoral implant described herein.

FIG. 23 shows schematically one embodiment of a hand prosthesis described herein.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases. As used herein “a” and “an” refer to one or more.

As used herein, the terms “comprising,” “comprise” or “comprised,” and variations thereof, are open ended and do not exclude the presence of other elements not identified. In contrast, the term “consisting of” and variations thereof is intended to be closed, and excludes additional elements in anything but trace amounts. A “copolymer consisting essentially of” two or more monomers or residues means that the copolymer is produced from the stated two or more monomers or contains the stated two or more monomers and is prepared from no other monomers or contains no other residues in any quantity sufficient to substantially affect the biological properties of the composition.

Extracellular matrix (ECM) derived from mammalian tissues has been utilized to repair damaged or missing tissue and improve healing outcomes. More recently, processing of ECM into hydrogels has expanded the use of these materials to include platforms for 3-dimensional cell culture as well as injectable therapeutics that can be delivered by minimally invasive techniques and fill irregularly shaped cavities. At the cellular level, ECM hydrogels initiate a multifaceted host response that includes recruitment of endogenous stem/progenitor cells, regional angiogenesis, and modulation of the innate immune response. Unfortunately, little is known about the components of the hydrogel that drive these responses. We hypothesized that different components of ECM hydrogels could play distinctive roles in stem cell and macrophage behavior. Utilizing a well-characterized ECM hydrogel derived from urinary bladder matrix (UBM), we separated the soluble and structural components of UBM hydrogel and characterized their biological activity. Perivascular stem cells migrated toward and reduced their proliferation in response to both structural and soluble components of UBM hydrogel. Both components also altered macrophage behavior but with different fingerprints. Soluble components increased phagocytosis with an IL-1RA^{high}, TNFα^{low}, IL-1β^{low} secretion profile. Structural components decreased phagocytosis with a PGE2^{high} PGF2α^{high}, TNFα^{low}, IL-1β^{low} secretion profile. Collectively, these findings demonstrate that soluble and structural components of ECM hydrogels contribute to the host response but through different mechanisms.

As used herein, the terms “extracellular matrix” and “ECM” refer to a natural scaffolding for cell growth. Natural ECMs (ECMs found in multicellular organisms, such as mammals and humans) are complex mixtures of structural and non-structural biomolecules, including, but not limited to, collagens, elastins, laminins, glycosaminoglycans, proteoglycans, antimicrobials, chemoattractants, cytokines, and growth factors. In mammals, ECM often comprises about 90% collagen, in its various forms. The composition and structure of ECMs vary depending on the source of the tissue. For example, small intestine submucosa (SIS), urinary bladder matrix (UBM), liver stroma ECM, and dermal ECM each differ in their overall structure and composition due to the unique cellular niche needed for each tissue.

As used herein, the terms “intact extracellular matrix” and “intact ECM” refers to an extracellular matrix that retains activity of at least a portion of its structural and non-structural biomolecules, including, but not limited to, collagens, elastins, laminins, glycosaminoglycans, proteoglycans, antimicrobials, chemoattractants, cytokines, and/or growth factors, such as, without limitation comminuted ECM as described herein. The activity of the biomolecules within the ECM can be removed chemically or mechanically, for example, by cross-linking and/or by dialyzing the ECM. Intact ECM essentially has not been cross-linked and/or dialyzed, meaning that the ECM has not been subjected to a dialysis and/or a cross-linking process, or conditions other than decellularization processes or processes that occur as part of storage and handling of ECM prior to solubilization, as described herein. Thus, ECM that is substantially cross-linked and/or dialyzed (in anything but a trivial manner which does not substantially affect the gelation and functional characteristics of the ECM in its uses described herein) is not considered to be “intact”.

ECM, for example intact ECM is typically prepared by the decellularization of tissues prior to use. As indicated above, decellularization is performed to prevent a pro-inflammatory response. As such, a decellularized ECM product or a decellularized intact ECM product is used herein to refer to ECM material that is decellularized to the extent that a pro-inflammatory response, and thus growth of fibrotic tissue is not is not elicited to any substantial degree in favor of constructive remodeling; for example and without limitation, resulting in a M2 macrophage phenotype rather than an M1 macrophage phenotype, responses characteristic of the M2 phenotype rather than responses characteristic of an M1 phenotype, and/or resulting in a greater proportion of M2 macrophage as compared to M1 macrophage in response to implantation of the ECM material in a mammal.

By “bio compatible”, it is meant that a device, scaffold composition, etc. is essentially, practically (for its intended use) and/or substantially non-toxic, non-injurous or non-inhibiting or non-inhibitory to cells, tissues, organs, and/or organ systems that would come into contact with the device, scaffold, composition, etc.

In general, the method of preparing an ECM-derived gel requires the isolation of ECM from an animal of interest and from a tissue or organ of interest. In certain embodiments, the ECM is isolated from mammalian tissue. As used herein, the term “mammalian tissue” refers to tissue derived from a mammal, wherein tissue comprises any cellular component of an animal. For example and without limitation, tissue can be derived from aggregates of cells, an organ, portions of an organ, or combinations of organs. In certain embodiments, the ECM is isolated from a vertebrate animal, for example and without limitation, human, monkey, pig, cattle, and sheep. In certain embodiments, the ECM is isolated from any tissue of an animal, for example and without limitation, urinary bladder, liver, small intestine, esophagus, pancreas, dermis, and heart. In one embodiment, the ECM is derived from a urinary bladder. The ECM may or may not include the basement membrane portion of the ECM. In certain embodiments, the ECM includes at least a portion of the basement membrane. The ECM may or may not retain some of the cellular elements that comprised the original tissue such as capillary endothelial cells or fibrocytes. In one embodiment, the ECM is derived from dermal tissue.

As used herein, the term “derive” and any other word forms or cognates thereof, such as, without limitation, “derived” and “derives”, refers to a component or components obtained from any stated source by any useful method. For example and without limitation, an ECM-derived gel refers to a gel comprised of components of ECM obtained from any tissue by any number of methods known in the art for isolating ECM. In another example, mammalian tissue-derived ECM refers to ECM comprised of components of a particular mammalian tissue obtained from a mammal by any useful method.

The methods described herein involve preparation of an ECM gel. The ECM-derived gel is reverse gelling, or can be said to exhibit reverse thermal gelation, in that it forms a gel (sol to gel transition) upon an increase in temperature. The lower critical solution temperature (LCST) in a reverse gel is a temperature below which a reverse-gelling polymer is soluble in its solvent (e.g. water or an aqueous solvent). As the temperature rises above the LCST in a reverse gel, a hydrogel is formed. The general concept of reverse gelation of polymers and its relation to LCST are broadly known in the chemical arts. The ECM gels described herein are prepared, for example from decellularized, intact ECM as described below, by digestion of the ECM material with an acid protease, neutralization of the material to form a pre-gel, inserting a polymeric mesh into the pre-gel and then raising the temperature of the pre-gel above the LCST of the pre-gel to cause the pre-gel to gel. As used herein, the term “gel” includes hydrogels. The transition temperature for acid-protease-digested from solution to gel is typically within the range of from 10° C. to 40° C. and any increments or ranges therebetween, for example from 20° C. to 35° C. For example, the pre-gel can be warmed to 37° C. to form a hydrogel.

Tissue for preparation of ECM and ECM-derived pre-gel solutions and gels can be harvested in a large variety of ways and once harvested, a variety of portions of the harvested tissue may be used. For example and without limitation, in one embodiment, the ECM is isolated from harvested porcine urinary bladder to prepare urinary bladder matrix (UBM). Excess connective tissue and residual urine are removed from the urinary bladder. The tunica serosa, tunica muscularis externa, tunica submucosa and most of the muscularis mucosa can be removed mechanical abrasion or by a combination of enzymatic treatment, hydration, and abrasion. Mechanical removal of these tissues can be accomplished by abrasion using a longitudinal wiping motion to remove the outer layers (particularly the abluminal smooth muscle layers) and even the luminal portions of the tunica mucosa (epithelial layers). Mechanical removal of these tissues is accomplished by removal of mesenteric tissues with, for example, Adson-Brown forceps and Metzenbaum scissors and wiping away the tunica muscularis and tunica submucosa using a longitudinal wiping motion with a scalpel handle or other rigid object wrapped in moistened gauze. The epithelial cells of the tunica mucosa can also be dissociated by soaking the tissue in a de-epithelializing solution, for example and without limitation, hypertonic saline. The resulting UBM comprises basement membrane of the tunica mucosa and the adjacent tunica propria, which is further treated with peracetic acid, lyophilized and powdered.

In another embodiment, dermal tissue is used as the source of ECM. Dermal tissue may be obtained from any mammalian source, such as human, monkey, pig, cow and sheep. In one embodiment, the source is porcine. Porcine skin from the dorsolateral flank of market weight pigs immediately can be harvested and processed by soaking in water or distilled water. All samples were then delaminated to remove subcutaneous fat, connective tissue and the epidermis. The harvested sheets of porcine dermis are immediately frozen at ˜80° C.

Dermis sections may be decellularized with 0.25% Trypsin/1% Triton X-100 (i.e. no SDS) on a vortex shaker at 300 RPM at room temperature in the following solutions: 0.25% trypsin for 6 hours, 1×; deionized water, 15 minutes, 3×; 70% ethanol, 10 to 12 hours, 1×; 3% H₂O₂, 15 minutes, 1×, deionized water, 15 minutes, 2×; 1% Triton X-100 in 0.26% EDTA/0.69% Tris, 6 hours, 1× and then overnight, 1×; deionized water, 15 minutes, 3×; 0.1% peracetic acid/4% ethanol, 2 hours, 1×; PBS, 15 minutes, 2×; and finally deionized water, 15 minutes, 2×. Dermis sheets are then lyophilized and subsequently reduced to particulate form using a Waring blender and a Wiley Mill with a #20 mesh screen.

In another embodiment, the epithelial cells can be delaminated first by first soaking the tissue in a de-epithelializing solution such as hypertonic saline, for example and without limitation, 1.0 N saline, for periods of time ranging from 10 minutes to 4 hours. Exposure to hypertonic saline solution effectively removes the epithelial cells from the underlying basement membrane. The tissue remaining after the initial delamination procedure includes epithelial basement membrane and the tissue layers abluminal to the epithelial basement membrane. This tissue is next subjected to further treatment to remove the majority of abluminal tissues but not the epithelial basement membrane. The outer serosal, adventitial, smooth muscle tissues, tunica submucosa and most of the muscularis mucosa are removed from the remaining de-epithelialized tissue by mechanical abrasion or by a combination of enzymatic treatment, hydration, and abrasion.

In one embodiment, the ECM is prepared by abrading porcine bladder tissue to remove the outer layers including both the tunica serosa and the tunica muscularis using a longitudinal wiping motion with a scalpel handle and moistened gauze. Following eversion of the tissue segment, the luminal portion of the tunica mucosa is delaminated from the underlying tissue using the same wiping motion. Care is taken to prevent perforation of the submucosa. After these tissues are removed, the resulting ECM consists mainly of the tunica submucosa.

Following isolation of the tissue of interest, decellularization is performed by various methods, for example and without limitation, exposure to hypertonic saline, peracetic acid, Triton-X or other detergents. Sterilization and decellularization can be simultaneous. For example and without limitation, sterilization with peracetic acid, described above, also can serve to decellularize the ECM. As indicated above, decellularized ECM is decellularized to an extent that avoids elicitation of a pro-inflammatory (e.g., M1 macrophage phenotype) response, and means that there is a sufficiently low concentration or amounts of DNA, phospholipid, and/or mitochondrial material in the resulting solution. In certain embodiments, the ECM is considered decellularized when there is less than 50 ng DNA/mg ECM in the decellularized ECM, digest solution and/or resulting pre-gel solution. In other embodiments, the ECM is considered decellularized when there is less than 750 nmol phospholipids/g ECM in the solution and/or resulting pre-gel solution.

Decellularized ECM can then be dried, either lyophilized (freeze-dried) or air dried. The ECM is optionally comminuted at some point prior to enzymatic digestion, for example prior to or after decellularization and/or drying. Dried ECM can be comminuted by methods including, but not limited to, tearing, milling, cutting, grinding, and shearing. The comminuted ECM can also be further processed into a powdered form by methods, for example and without limitation, such as grinding or milling in a frozen or freeze-dried state.

Non-limiting additional examples of extracellular matrix preparations are described in U.S. Pat. Nos. 4,902,508; 4,956,178; 5,281,422; 5,352,463; 5,372,821; 5,554,389; 5,573,784; 5,645,860; 5,711,969; 5,753,267; 5,762,966; 5,866,414; 6,099,567; 6,485,723; 6,576,265; 6,579,538; 6,696,270; 6,783,776; 6,793,939; 6,849,273; 6,852,339; 6,861,074; 6,887,495; 6,890,562; 6,890,563; 6,890,564; and 6,893,666. In certain embodiments, the ECM is isolated from a vertebrate animal, for example and without limitation, from a warm-blooded mammalian vertebrate animal including, but not limited to, human, monkey, pig, cow and sheep. The ECM can be derived from any organ or tissue, including without limitation, urinary bladder, intestine, liver, esophagus and dermis. In one embodiment, the ECM is isolated from a urinary bladder. The ECM may or may not include the basement membrane portion of the ECM. In certain embodiments, the ECM includes at least a portion of the basement membrane. In other embodiments, the ECM is isolated from dermal tissue.

In addition to producing ECM as described above, commercially-available ECM preparations can also be used in the devices, compositions and methods described herein. In one embodiment, the ECM is derived from small intestinal submucosa or SIS. Commercially available preparations include, but are not limited to, Surgisis™, Surgisis-ES™, Stratasis™, and Stratasis-ES™ (Cook Urological Inc.; Indianapolis, Ind.) and GraftPatch™ (Organogenesis Inc.; Canton Mass.). In another embodiment, the ECM is derived from dermis. Commercially available preparations include, but are not limited to Pelvicol™ (sold as Permacol™ in Europe; Bard, Covington, Ga.), Repliform™ (Microvasive; Boston, Mass.) and Alloderm™ (LifeCell; Branchburg, N.J.). In another embodiment, the ECM is derived from urinary bladder. Commercially available preparations include, but are not limited to UBM (Acell Corporation; Jessup, Md.).

As used herein, the term “comminute” and any other word forms or cognates thereof, such as, without limitation, “comminution” and “comminuting”, refers to the process of reducing larger particles into smaller particles, including, without limitation, by grinding, blending, shredding, slicing, milling, cutting, shredding. ECM can be comminuted while in any form, including, but not limited to, hydrated forms, frozen, air-dried, lyophilized, powdered, sheet-form.

In order to prepare solubilized ECM tissue, comminuted ECM is digested with an acid protease in an acidic solution to form a digest solution. As used herein, the term “acid protease” refers to an enzyme that cleaves peptide bonds, wherein the enzyme has increased activity of cleaving peptide bonds in an acidic pH. For example and without limitation, acid proteases include pepsin and trypsin and mixtures thereof.

As an example, the digest solution of ECM is kept at a constant stir for a certain amount of time at room temperature. The ECM digest can be used immediately or be stored at −20° C. or frozen at, for example and without limitation, −20° C. or −80° C. In certain embodiments, the ECM digest is snap frozen in liquid nitrogen. To form a “pre-gel” solution, the pH of the digest solution is raised to a pH between 7.2 and 7.8. The pH can be raised by adding one or more of a base or an isotonic buffered solution, for example and without limitation, NaOH or PBS at pH 7.4. The method optionally does not include a dialysis step prior to gelation, yielding a more-complete ECM-like matrix that typically gels at 37° C. more slowly than comparable collagen or dialyzed ECM preparations. In certain embodiments, dialysis, or similar methods, are not used. The gel therefore retains more of the qualities of native ECM due to retention of many native soluble factors, such as, without limitation, cytokines. These factors contribute to chemoattraction of cells and proper rearrangement of tissue at the site of injury, rather than fibrous response that leads to unwanted scarring. In other embodiments, the ECM is dialyzed prior to gelation to remove certain soluble components.

As used herein, the term “isotonic buffered solution” refers to a solution that is buffered to a pH between 7.2 and 7.8, e.g., pH 7.4, and that has a balanced concentration of salts to promote an isotonic environment. As used herein, the term “base” refers to any compound or a solution of a compound with a pH greater than 7. For example and without limitation, the base is an alkaline hydroxide or an aqueous solution of an alkaline hydroxide. In certain embodiments, the base is NaOH or NaOH in PBS.

This “pre-gel” solution can, at that point be incubated at a suitably warm temperature, for example and without limitation, at about 37° C. to gel.

In order to separate the structural from soluble components of the resultant hydrogel, the hydrogel is centrifuged at a sufficient g-force and for a sufficient time to separate the solution and structural components of the hydrogel. By solution in the context of this separation method, it is referred to the resultant aqueous solution and constituents dissolved or otherwise remaining in the aqueous solution after centrifugation at 25,000×g (25,000 times gravity) for 30 minutes.

As used herein, a “polymer” is a compound formed by the covalent joining of smaller molecules, which are referred to herein as monomers before incorporation into the polymer and residues, or polymer subunits, after incorporation into a polymer. A “copolymer” is a polymer comprising two or more different residues. Non-limiting examples of monomers, in the context of the copolymers described herein, include: acrylic or acrylamide monomers, acrylic N-hydroxysuccinimide ester monomers, N-hydroxysuccinimide methacrylate monomers, acrylate or methacrylate forms of N-acryloxy succinimide (NAS) monomers, hydroxyethyl methacrylate monomers, methacrylate monomers, acrylate or methacrylate forms of lactide monomers, and acrylate or methacrylate forms of trimethylene carbonate (TMC) monomers. A monomer may be a macromer prepared from smaller monomers. Polymers can be synthetic or natural, meaning they are man-made or found in nature. Collagen is an example of a natural polymer.

Provided herein is a method of preparing one or more biologically active fractions of ECM useful for modulating chemotaxis, immune response and proliferation of stem cells. The method comprises: partially or completely digesting with an acid protease, such as pepsin, decellularized ECM material prepared from a tissue; neutralizing the digested ECM material to a pH of 7.0-8.0, e.g., 7.2-7.8 or 7.4; gelling the neutralized, digested ECM material at a temperature above its Lower Critical Solution Temperature; centrifuging the gelled ECM material to produce a pellet and a supernatant; and separating the supernatant and the pellet thereby separating a structural and a soluble fraction of the ECM material. The structural and supernatant fractions produce different immune responses, with the structural components, favoring an upregulation of COX2 and prostaglandins, and the soluble components suppressing the classic inflammatory response while increasing the phagocytic activity of macrophages. To produce a structural fraction including structural ECM components, the pellet is dispersed, for example by homogenization, into a solution, e.g., an aqueous solution, such as water, saline, isotonic buffer, PBS or cell culture media, such as serum-free media. To produce a soluble fraction including soluble ECM components, first any remaining structural components are optionally removed by precipitation by adding salts (e.g., salting out, as is broadly known). The supernatant is then concentrated by drying, for example by spraying or lyophilization, and then can be reconstituted in a solution, e.g., an aqueous solution, such as water, saline, isotonic buffer, PBS or cell culture media, such as serum-free media. Lyophilization may occur at room temperature or at below room temperature, for example at 0° C., −10° C., −20° C., −30° C., and lower. When the soluble fraction is reconstituted, it is typically reconstituted to a fraction of the original volume of the supernatant, for example to <10%, 10%, 20%, 25%, 30%, 40% or 50% of the original volume of the supernatant.

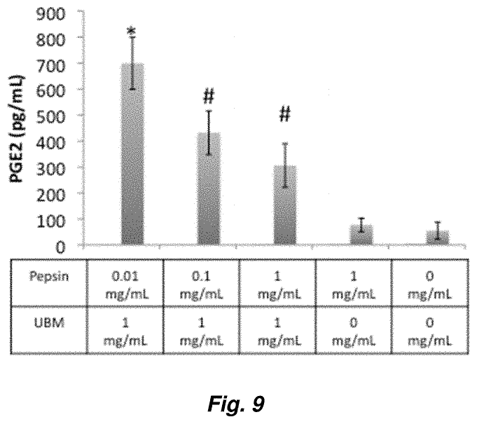

As indicated in Example 2 below, when preparing the ECM hydrogel, the digestion of the ECM material with the acid protease is in one embodiment, partial. Partial digestion preserved the structural components that elicit increased prostaglandin production, which is believed to be, or at least include, based on Example 3, hyaluronic acid. For example, in one embodiment, the decellularized ECM material is digested less completely than a digestion of 1 mg/mL lyophilized, powdered ECM material with 1 mg/mL pepsin in 0.01 M HCl for 48 hours. Alternately, in another embodiment, the decellularized ECM material is digested less completely than a digestion of 10 mg/mL lyophilized, powdered ECM material with 1 mg/mL pepsin in 0.01 M HCl for 48 hours. This degree of digestion can be determined by comparison on a gel, such as in FIG. 10, panel (A), or by ascertaining the degree of degradation of hyaluronic acid, for example by Western blot (anti-hyaluronic acid antibodies are commercially-available from multiple sources) or chromatographic methods, as are broadly known. For example in a partial digestion, hyaluronic acid is digested less than 50%, 40%, 30%, 25%, 20% or 10%.

In use, the ECM fractions described herein can be used to elicit a particular response. The compositions are applied either topically, for example to the skin, respiratory tract, mucosa or eye, or parenterally, for example in a wound, transplant or implant to elicit a response, such as chemotaxis, cell differentiation, or a particular immune response, such as a macrophage M2 response, such as increased phagocytosis and lowered classical inflammation in the case of administration of the soluble components or increased COX2 and prostaglandin activity, and establishing a pro-reconstruction environment for tissue infiltration and growth. The compositions may be applied or administered in a variety of ways, either as a dry, e.g., lyophilized powder, a solution, a gel, etc. The composition can be administered by itself, or with a device or composition. For example, the composition can be absorbed into, adsorbed onto, mixed into or otherwise co-administered with a cell-growth scaffold, such as an isotropic or anisotropic mass of fibers of synthetic and/or natural polymer(s), such as an electrodeposited, wet or dry spun, 3D printed, molded, or otherwise formed polymeric structure prepared from biocompatible polymeric materials, as are broadly known in the regenerative medical field, such as collagen, polyester, polyurethane, poly(ester urethane) urea, and poly(ether ester urethane) urea copolymers, and other suitable polymeric materials, such as are disclosed, for example and without limitation in U.S. Pat. Nos. 8,535,719; 8,673,295; 8,889,791; 8,974,542 and 9,023,972. The compositions described herein also can be mixed into polymeric compositions prior to or along with deposition of polymeric fibers or formation of structures. The compositions described herein can be sprayed onto, painted onto, or otherwise applied to a structure. In one embodiment, a composition as described herein is applied to and delivered from an ECM material, such as any commercial ECM material, such as those described above.

In a further embodiment, either the soluble or structural ECM component is added to an acid-protease digested pre-gel ECM composition, such as the composition described above in reference to preparation of the structural and soluble ECM components. The soluble or structural ECM component is added to the pre-gel ECM composition at any point prior to gelation, so as to increase the specific activity of one component of the ECM gel. After mixing of the components, the pre-gel is heated to a temperature above the LCST of the composition to produce a hydrogel. As described, for example and without limitation in U.S. Pat. No. 8,361,503, the hydrogel is useful as an injectable, or otherwise formable or moldable cell-growth scaffold or matrix for treatment of implantable devices, such as prostheses.

Likewise, the compositions described herein can be applied to or incorporated into, by any suitable method, a non-woven material, such as a bandage, a suture, an implant, such as a ceramic, metal, or polymeric implant, for example a prosthesis, artificial or otherwise-modified vessel, a valve, an intraocular lens, a tissue transplant or implant.

As used herein, the term “coat”, and related cognates such as “coated” and “coating,” refers to a process comprising of covering an inorganic structure with a composition described herein. For example and without limitation, coating of an inorganic structure with ECM-derived gel can include methods such as pouring, embedding, layering, dipping, spraying. Ultrasonication may be used to aid in coating of an inorganic structure with the ECM-derived gel. As used herein, the term “ultrasonication” refers to the process of exposing ultrasonic waves with a frequency higher than 15 kHz and lower than 400 kHz.

In another embodiment, the composition is coated onto a biocompatible structural material, such as a metal, an inorganic calcium compound such as calcium hydroxide, calcium phosphate or calcium carbonate, or a ceramic composition. Non-limiting examples of suitable metals are cobalt-chrome alloys, stainless steel alloys, titanium alloys, tantalum alloys, titanium-tantalum alloys, which can include both non-metallic and metallic components, such as molybdenum, tantalum, niobium, zirconium, iron, manganese, chromium, cobalt, nickel aluminum and lanthanum, including without limitation, CP Ti (commercially pure titanium) of various grades or Ti 6Al4V (90% wt. Ti, 6% wt. Al and 4% wt. V), stainless steel 316, Nitinol (Nickel-titanium alloy), titanium alloys coated with hydroxyapatite. Metals are useful due to high strength, flexibility, and biocompatibility. Metals also can be formed into complex shapes and many can withstand corrosion in the biological environments, reduce wear, and not cause damage to tissues. In one non-limiting example, the metal is femoral or acetabular component used for hip repair. In another example, the metal is a fiber or other protuberance used in permanent attachment of a prosthesis to a patient. Other compositions, including ceramics, calcium compounds, such as, without limitation, aragonite, may be preferred, for example and without limitation, in repair of or re-shaping of skeletal or dental structures. Combinations of metal, ceramics and/or other materials also may prove useful. For instance, a metal femoral component of a hip replacement may comprise a ceramic ball and/or may comprise a plastic coating on the ball surface, as might an acetabular component.

Metals, as well as other materials, as is appropriate, can be useful in its different forms, including but not limited to wires, foils, beads, rods and powders, including nanocrystalline powder. The composition and surface of metals or other materials can also be altered to ensure biocompatibility, such as surface passivation through silane treatments, coating with biocompatible plastics or ceramics, composite metal/ceramic materials. The materials and methods for their employment are well-known in the field of the present invention.

A difficulty with using metal inserts to repair a patient's skeletal structure is that the inserts must be anchored/attached to existing skeletal parts. Traditional methods employed cement and/or screws. In the case of prostheses, the prostheses are not connected to a patient's tissue except, typically, by cementing. Therefore, it is desirable to biologically attach a patient's tissue to a medical device. This may be accomplished by coating surfaces of the implant with a composition as described herein, which will facilitate in-growth of tissue and thus attachment of the device. A variety of porous structures can be attached to the implant to create a scaffold into which the composition, such as a gel comprising one of the structural or soluble ECM compositions described herein, and later cells or other tissue (e.g., bone) can infiltrate. Structures include, without limitation: woven or non-woven mesh, sponge-like porous materials, fused beads, etc. The porous scaffold will facilitate formation of a strong bond between living tissue, including bone, and the device. The “pores” of the porous scaffold may be of any size that will permit infiltration of a gel, optionally facilitated by ultrasound or other treatments that would assist in permeation of the gel, and later cells or other biological materials, such as bone, cartilage, tendons, ligaments, fascia or other connective tissue, into the scaffolding. In one embodiment, metal fibers are attached to the device, and the metal fibers are coated with an ECM composition as described herein, thereby permitting in-growth of tissue within the fibers. In a second embodiment, a matrix of small beads is welded or otherwise attached to a surface of the device and an ECM composition as described herein is coated onto the bead matrix, facilitating in-growth of tissue among the beads. In one example, a device contains a protuberance of fibers, which can be inserted inside a bone, permitting fusion of the metal fibers with the bone. In one embodiment, the ECM composition as described herein is seeded and incubated with a suitable cell population, such as autologous osteoblasts, to facilitate bone in-growth.

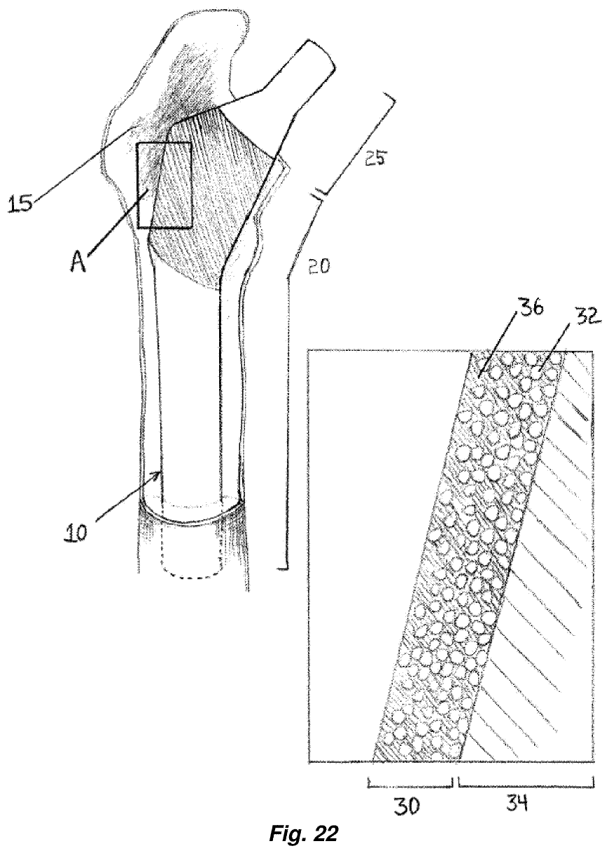

The ECM composition as described herein can be used to coat, without limitation, a femoral implant, or a prosthesis of the hand. FIG. 22 shows schematically one embodiment of a device 10 inserted into a femur 15 in a hip replacement procedure. FIG. 22 illustrates device 10, showing an insert portion 20 for insertion into femur 15, and an extension 25 into which a ball (not shown) is screwed or otherwise inserted. Device 10 comprises a porous coating 30 of, for example and without limitation, metal beads welded onto the device 10. Region A in FIG. 22 shows a magnified view of coating 30 of device 10. Beads 32 are welded to metal surface 34 of device 10. ECM gel 36 is coated onto and between beads 32. Bone tissue growth into beads 32 is facilitated by the presence of the ECM gel 36.

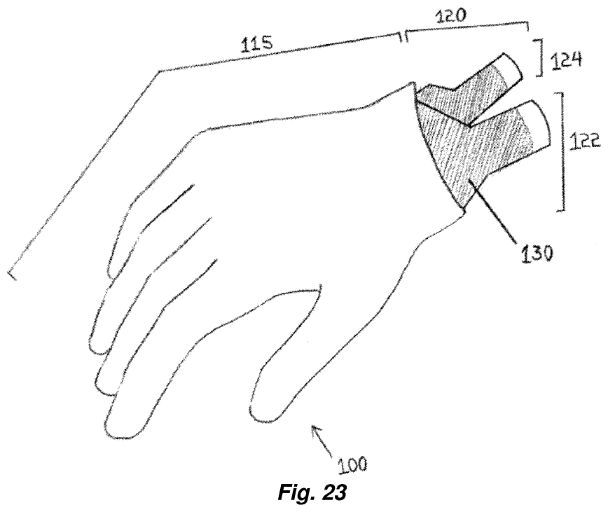

A prosthesis might be anchored into bone in a like manner using an insert having a porous coating, with the porous coating extending to the limits of where attachment to a patient's tissue is desired. As an example, shown in FIG. 23, a hand prosthesis 100 comprises an external portion 115 and an internal portion 120, which comprises a radius insert portion 122 and an ulnar insert portion 124. Porous coating 130 extends from insert portions 122 and 124 for attachment to bone, to the beginning of external portion 115, permitting attachment of dermis and intermediary tissue between the bones and dermis.

Any useful cytokine, chemoattractant, drug or cells can be mixed into, mixt with, co-applied or otherwise combined with any composition as described herein. For example and without limitation, useful components include growth factors, interferons, interleukins, chemokines, monokines, hormones, angiogenic factors, drugs and antibiotics. Cells can be mixed into the composition or can be included on or within a substrate such as a biological scaffold, combined with the composition. In either case, when the substrate is seeded with cells, the cells can be grown and/or adapted to the niche created by incubation in a suitable medium in a bioreactor or incubator for a suitable time period to optimally/favorably prepare the composition for implantation in a patient. The substrate can be seeded with cells to facilitate in-growth, differentiation and/or adaptation of the cells. For example and without limitation, the cells can be autologous or allogeneic with respect to the patient to receive the composition/device comprising the gel. The cells can be stem cells or other progenitor cells, or differentiated cells. In one example, a layer of dermis obtained from the patient is seeded on a mold, for use in repairing damaged skin and/or underlying tissue.

As used herein, the terms “drug” and “drugs” refer to any compositions having a preventative or therapeutic effect, including and without limitation, antibiotics, peptides, hormones, organic molecules, vitamins, supplements, factors, proteins and chemoattractants.

As used herein, the terms “cell” and “cells” refer to any types of cells from any animal, such as, without limitation, rat, mice, monkey, and human. For example and without limitation, cells can be progenitor cells, such as stem cells, or differentiated cells, such as endothelial cells, smooth muscle cells. In certain embodiments, cells for medical procedures can be obtained from the patient for autologous procedures or from other donors for allogeneic procedures.

In a further embodiment, a commercial kit is provided comprising a composition described herein. A kit comprises suitable packaging material and the composition. In one non-limiting embodiment, the kit comprises a liquid or dried structural or soluble ECM fraction or components in a vessel, which may be the packaging, or which may be contained within packaging. The vessel may be a vial, syringe, tube or any other container suitable for storage and transfer in commercial distribution routes of the kit. Likewise, a product, such as a device, gel, scaffolding, suture, prosthetic, mesh, etc. including one or both of the soluble or structural compositions described herein may be packaged appropriately for commercial distribution.

### Example 1

At a molecular level, ECM hydrogels consist of both structural and soluble phases that contain unique molecular fingerprints. The structural phase of ECM hydrogels includes a number of proteins and proteoglycans including collagens, elastin, laminin, fibronectin, hyaluronan, and heparan. The soluble phase of the scaffold contains some full-length proteins such as growth factors (e.g. bFGF, VEGF, IGF, TGFβ) and matricellular proteins (e.g. SPARC, tenascin, osteopontin, thrombospondin) as well as cryptic peptide fragments generated from partial proteolysis of the aforementioned structural and soluble proteins. Despite extensive studies on the biochemical composition and mechanical properties of ECM hydrogels, little is known about the roles that these structural and soluble components play in the host remodeling response.

The objective of the present study was to separate the soluble components of an ECM hydrogel from the structural components in an effort to distinguish which components retain some or any biological activity. The well-characterized hydrogel of the biologic scaffold referred to as urinary bladder matrix (UBM) was utilized as a model system. The structural and soluble components of UBM hydrogel were fractionated and tested for their ability to modulate stem cell and macrophage behavior—two critical cell types in the host response to ECM. The components of UBM hydrogel were tested for their ability to modulate the chemotactic and proliferative activity of human perivascular stem cells (PSCs). Additionally, the components were tested for their ability to modulate macrophage behavior by examining their effects on the phagocytic ability and secretion profile of THP1 human macrophages.

### Materials and Methods

### Reagents

All chemical reagents were purchased from Sigma-Aldrich (St. Louis, Mo.) unless otherwise specified. All cell culture media and reagents were purchased from Life Technologies (Carlsbad, Calif.) unless otherwise specified. All chemicals used were reagent grade or better.

### Urinary Bladder Matrix Preparation

Porcine urinary bladders were acquired from market weight pigs (110-130 kg) as a byproduct of routine commercial production. The extracellular matrix from this tissue referred to as UBM was prepared as previously described [Freytes D O, Tullius R S, Badylak S F. Effect of storage upon material properties of lyophilized porcine extracellular matrix derived from the urinary bladder. J Biomed Mater Res B Appl Biomater 2006; 78:327-33]. Briefly, the tunica serosa, tunica muscularis externa, tunica submucosa, and most of the tunica muscularis mucosa were mechanically removed and the luminal urothelial cells of the tunica mucosa were dissociated by rinsing in sterile water. The remaining tissue consisted of the basement membrane, the subjacent tunica propria of the tunica mucosa, and any resident cells in those layers. The matrix was decellularized by agitation in 0.1% peracetic acid with 4% ethanol for 2 hours at high speed followed by extensive rinsing with phosphate-buffered saline (PBS) and sterile water. Decellularization was verfied using 4′-6-diamidino-2-phenylindole (DAPI, Fisher Scientific, Waltham, Mass.) nuclear staining and quantification of remnant DNA [Crapo P M, Gilbert T W, Badylak S F. An overview of tissue and whole organ decellularization processes. Biomaterials 2011; 32:3233-43]. The UBM was then lyophilized into a dry sheet and either milled into particulates using a Wiley Mill with a #60 mesh screen [Gilbert T W, Stolz D B, Biancaniello F, Simmons-Byrd A, Badylak S F. Production and characterization of ECM powder: implications for tissue engineering applications. Biomaterials 2005; 26:1431-5] or left as a dry sheet.

### Pepsin Mediated ECM Solubilization

UBM was enzymatically digested as previously described [Freytes D O, Martin J, Velankar S S, Lee A S, Badylak S F. Preparation and rheological characterization of a gel form of the porcine urinary bladder matrix. Biomaterials 2008; 29:1630-7] with pepsin by mixing lyophilized, powdered UBM (10 mg/mL) and pepsin (1 mg/mL) in 0.01 M HCl (pH 2.0). This solution was stirred at room temperature for 48 hours. After stirring, the UBM slurry was neutralized to a pH of 7.4 in 1×PBS (137 mM NaCl, 2.7 mM KCl, 12 mM Phosphate, Fisher Scientific, Waltham, Mass.) to inactivate the pepsin and prepare the material for cell culture assays. A solution of pepsin (1 mg/mL) in 0.01M HCl, treated in the same fashion as the UBM sample, served as the control condition for all experiments. All materials were stored at −80° C. until use.

### Fractionation of Digested UBM

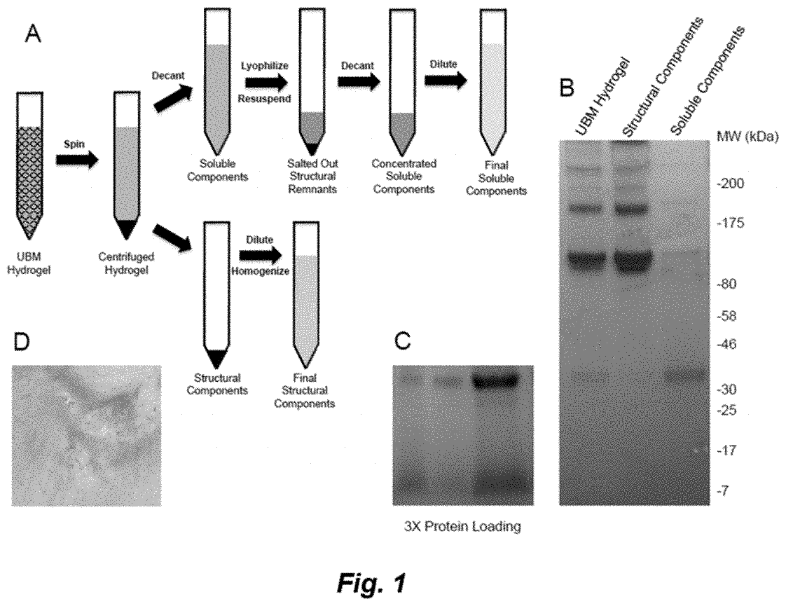

Neutralized UBM digest was incubated at 37° C. to induce gelation. The UBM hydrogel was then centrifuged at 25,000×g for 30 minutes to compress the insoluble, structural components of the scaffold into a pellet, leaving a clear supernatant above the pellet (FIG. 1A). The gel pellet containing the structural components was collected and resuspended to the starting volume in 1×PBS. Due to the insolubility of the gel pellet, the gel pellet suspension was vigorously pipetted through a 10 μL pipet tip to homogenize the material as much as possible. The homogenized suspension was stored at −80° C. until use. The clear supernatant containing the soluble components was removed and lyophilized to dryness. The dried supernatant was rehydrated in 10% of its original volume with sterile water to drive the PBS concentration from 1× to 10×. The rehydrated soluble components were centrifuged at 20,000×g to clarify the solution. The supernatant from this final spin was removed, diluted to the starting volume, and stored at −80° C. until use. Dilution to the starting volume for both fractionated components allowed direct comparison of the biological activity of the fractions using the same dilution factor for all materials.

### SDS PAGE and Protein Quantification

UBM Digest, Structural, and Soluble components were diluted 1:1 in 2× Laemmli Sample Buffer (Bio-Rad, Hercules, Calif.) and boiled at 95° C. for 8 minutes. Samples were diluted and tested for protein concentration using the Pierce BCA Protein Assay (Thermo Fisher, Waltham, Mass.) according to the manufacturer's instructions. Absorbances were measured at the appropriate wavelength using a Molecular Devices SpectraMax M2 plate reader (Silicon Valley, Calif.) and concentrations were approximated using a bovine serum albumin (BSA) standard curve. Either 15 μg or 45 μg of each sample-based on protein concentration—was resolved on 4-20% SDS PAGE gels (Bio-Rad, Hercules, Calif.) and stained with Coomassie Blue R-250 (Fisher Scientific, Waltham, Mass.). Gels were imaged using the Protein Simple Red Imager (Protein Simple, Santa Clara, Calif.).

### Cell Culture

THP-1 human monocytes were obtained from the American Tissue Culture Collection (ATCC, Manassas, Va.) and maintained in RPMI, 10% FBS, 1% penicillin/streptomycin, 2 mM L-glutamine, and 1 mM sodium pyruvate in a humidified atmosphere at 37° C. with 5% CO2. For TNFα, PGE2, PGF2α, IL-1β, and IL-1RA experiments, 500,000 THP-1 cells/well were plated with 320 nM phorbol 12-myristate 13-acetate (PMA) for 24 hours to induce differentiation into macrophages. Adherent macrophages were washed in PBS and placed in fresh media, followed by a 24 hour incubation in fresh media to acquiesce. Human PSCs were isolated from blood vessels in fetal muscle according to the methods outlined by Crisan et al. [Purification and long-term culture of multipotent progenitor cells affiliated with the walls of human blood vessels: myoendothelial cells and pericytes. Methods Cell Biol 2008; 86:295-309; Purification and culture of human blood vessel-associated progenitor cells. Curr Protoc Stem Cell Biol 2008; Chapter 2: Unit 2B 1-2B 13; and A perivascular origin for mesenchymal stem cells in multiple human organs. Cell Stem Cell 2008; 3:301-13] and maintained as reported.

### Chemotaxis Assay

Chemotaxis assays were performed in standard chemotaxis chambers with 8 μm filters (Neuro Probe, Gaithersburg, Md.) coated with rat-tail collagen (BD Biosciences, San Jose, Calif.) as described previously [Reing J E, Zhang L, Myers-Irvin J, Cordero K E, Freytes D O, Heber-Katz E, et al. Degradation products of extracellular matrix affect cell migration and proliferation. Tissue Eng Part A 2009; 15:605-14]. Briefly, PSCs were grown to 80-90% confluence before overnight incubation in DMEM with 0.5% heat inactivated FBS. Cells were trypsinized and resuspended in plain DMEM and 30,000 cells were loaded onto the top well of the chemotaxis chamber which was separated by the filter from the lower well containing the treatment condition (580-5.8 μg/mL UBM digest, 160-1.6 μg/mL soluble components, and 340-3.4 μg/mL structural components). Chambers were place in a humidified atmosphere at 37° C. with 5% CO₂ for 3 hours. Migrated cells were stained with DAPI (Fisher Scientific, Waltham, Mass.), imaged using a Zeiss Axio-Observer Z.1 microscope (Oberkochen, Germany) with 10× objective and quantified with ImageJ (NIH, Bethesda, Md.). Digestion enzyme only control yielded 163+35 migrated cells. This baseline value was normalized to zero and changes in chemotaxis for all treatment groups were expressed as a fold change to this control.

### Proliferation Assay

PSCs were cultured at a concentration of 5,000 cells per well in a 96 well plate for 24 hours. Following 24 hours, 580-5.8 μg/mL UBM digest, 160-1.6 μg/mL soluble components, or 340-3.4 μg/mL structural components were added to the wells along with 5-bromo-2′-deoxyuridine (BrdU; final concentration: 10 μM). Treated cell were incubated for 18 hours. The cells were then fixed with 95% methanol for 15 minutes and washed thoroughly with PBS. Cells were then incubated in 2M HCl for 30 minutes at 37° C. and washed repeatedly with PBS. Following these washes, the fixed cells were incubated in (0.01% Triton-X100, 0.01% Tween-20, 2% horse serum) blocking buffer for 1 hour at room temperature. Cells were then incubated in primary mouse anti-BrdU (1:1000, DSHB, G3G4-c, Iowa City, Iowa) overnight at 4° C. A donkey anti-mouse secondary antibody (1:300 Alexa Fluor 488, Life Technologies, Carlsbad, Calif.) was incubated for 45 minutes at room temperature. The cells were washed thoroughly with PBS and counterstained with DAPI. Whole well images were taken at 10× magnification using the mosaic function on the Zeiss Axio-Observer Z.1 microscope (Oberkochen, Germany) Cells positive for BrdU were then quantified using Cell Profiler[CellProfiler: image analysis software for identifying and quantifying cell phenotypes. Genome Biol 2006; 7:R100] and MATLAB (MathWorks, Natick, Mass.). Digestion enzyme only control contained 52+4 BrdU+ DAPI+ nuclei per field. This baseline value was normalized to zero and changes in proliferation for all treatment groups were expressed as a percent decrease from this control.

### Phagocytosis Assay

Macrophages prepared as described in the Cell Culture section were treated with UBM digest (290 μg/mL) or fractionated components (80 μg/mL soluble, 170 μg/mL structural) for 48 hours. After treatment cells were washed extensively with 1×PBS and placed in fresh media containing Fluoresbrite Latex Beads (1.0 μM) and incubated for 1 hour. Cells were then washed with 1×PBS and fixed with 4% PFA for 15 minutes. After fixation cells were washed in 1×PBS and treated with Accutase for 10 minutes to lift the cells. Lifted cells were transferred to Eppendorf tubes and washed in 1×PBS. Phagocytosis was quantified using a BD FACSAria II flow cytometer to count the number of phagocytosing and nonphagocytosing cells. Between 10,000 and 20,000 events were counted for each treatment. Three replicate treatments were completed for each experiment and data were expressed as a percentage of phagocytosing cells.

### Quantification of TNFα, PGE2, PGF2α, IL-1β, and IL1-RA by THP-1 Macrophages

Macrophages prepared as described in the Cell Culture section were treated with UBM digest (290 μg/mL) or fractionated components (80 μg/mL soluble, 170 μg/mL structural) for 48 (TNFα, IL-1β and IL-1RA), or 72 (PGE2 and PGF2α) hours. For TNFα and IL-1β, post-treatments with lipopolysaccharide (LPS, 100 ng/mL) were carried out for two hours. Culture supernatants were centrifuged at 20,000×g to pellet the cells and debris from the biologic scaffold treatments. Supernatants were harvested and frozen at −80° C. until the day of assay. Commercially available ELISA kits were used to determine the absolute quantities of TNFα (BD Bioscience, San Jose, Calif.), PGE2 (Enzo, Farmingdale, N.Y.), PGF2α (Enzo, Farmingdale, N.Y.), IL-1β (BD Bioscience, San Jose, Calif.), and IL1-RA (R&D Systems, Minneapolis, Minn.) according to the manufacturer's instructions. Detection limits for the kits were as follows: TNFα 70+3 pg/mL, PGE2 35+2 pg/mL, PGF2a 3+1 pg/mL, IL-1β 1.7+0.4 pg/mL, IL1-RA 12+3 pg/mL.

### In Vivo Evaluation

In vivo studies were conducted with a rodent partial thickness abdominal wall defect model [Badylak S, Kokini K, Tullius B, Simmons-Byrd A, Morff R. Morphologic study of small intestinal submucosa as a body wall repair device. J Surg Res 2002; 103:190-202; Valentin J E, Badylak J S, McCabe G P, Badylak S F. Extracellular matrix bioscaffolds for orthopaedic applications. A comparative histologic study. J Bone Joint Surg Am 2006; 88:2673-86; and Sicari B, Turner N, Badylak S F. An in vivo model system for evaluation of the host response to biomaterials. Methods Mol Biol 2013; 1037:3-25]. Briefly, female Sprague-Dawley rats at 250-300 g were anesthetized with 2.5-3% isofluorane and maintained with 2% isofluorane throughout the procedure. A 4 cm ventral midline skin incision was made and two 1 cm×1 cm partial thickness paramedian defects were created by removing the internal and external oblique muscles. The transversalis fascia and peritoneum were left intact as much as possible. The defects had a size-matched block of UBM hydrogel approximately 5 mm thick placed in the defect area. The skin incision was closed with 4-0 Vicryl (Ethicon, Somerville, N.J.) suture and the animals were allowed to recover with access to food and water ad libitum. Animals were survived for 3 and 7 days post implantation after which time, the defect area and the surrounding muscle tissue was explanted for histologic analysis.

### Immunolabeling of In Vivo Sections

Macrophages present in tissue sections were immunolabeled using previously described methods [Keane T J, Londono R, Carey R M, Carruthers C A, Reing J E, Dearth C L, et al. Preparation and characterization of a biologic scaffold from esophageal mucosa. Biomaterials 2013; 34:6729-37]. Briefly, paraffin embedded sections were washed with xylene and rehydrated slowly with an ethanol gradient. Antigen retrieval was carried out by heating the sections to 95° C. in 0.01 M citrate buffer (pH=6) for 25 min Tissue sections were washed in Tris-Buffered Saline Tween-20 (TBST) and incubated in blocking buffer consisting of 2% horse serum albumin, 1% BSA, 0.05% Tween-20, and 0.05% Triton X-100 in TBS for 1 hour. The primary antibodies, mouse anti-rat CD68 (1:100 AbD Serotec, Raleigh, N.C.), goat anti-rat CD206 (1:100 Santa Cruz, Dallas, Tex.), and rabbit anti-rat COX2 (1:250, Abcam, Cambridge, England) were diluted in blocking buffer, added to the sections and incubated overnight at 4° C. in a humidified chamber. After primary labeling, tissue sections were washed extensively in PBS and species appropriate secondary antibodies (donkey anti-rabbit Alexa-Fluor 488 (1:300), donkey anti-mouse Alex-Fluor 594(1:300), donkey anti-goat Alex-Fluor 594(1:300), Life Technologies, Carlsbad, Calif.)) were added and incubated for 1 hour at room temperature in a humidified chamber. Tissue sections were further washed with TBST and imaged at 32× magnification using a Nikon Eclipse E600 microscope (Chiyoda, Tokyo) with CRi Nuance FX multispectral imaging system (Cambridge, Mass.). Co-labeling experiments were carried out for CD68 and COX2, as well as CD206 and COX2. Representative images of each colabeled tissue section were collected.

### Statistics

Where appropriate, a one-way or two-way analysis of variance (ANOVA) was performed to determine significant differences with Student-Newman-Keuls post hoc tesing (P<0.05). Data and error bars are reported as mean+standard deviation unless otherwise specified.

### Results

The objective of this study was to determine the biological activity of the structural and soluble components of ECM hydrogels to better understand their role in host remodeling outcomes. To accomplish this objective, UBM hydrogels were fractionated into their soluble and structural components by centrifugation and salt precipitation. The effects of these fractions on stem cells and macrophages—two cell types that are critical to host remodeling—were investigated. The effects of structural and soluble components on stem cells were investigated by determining changes in isolated human PSC chemotaxis and proliferation. The effects of structural and soluble components on macrophages were investigated by examining changes in the secretion profile and phagocytic ability of THP1 human macrophages.

### Fractionation of UBM Digest

UBM hydrogels were crudely fractionated into soluble and structural components using the scheme described in FIG. 1A. Acid digestion of ECM scaffolds unfolds structural proteins allowing for the release of embedded soluble proteins and proteolysis of matricryptic peptides. Neutralization of UBM digest (pH 2.0 to pH 7.4) in 1×PBS allows the structural proteins to refold, forming a relatively stiff gel at 37° C. Centrifuging the gel at 25,000×g yielded a gel pellet containing the insoluble, structural scaffold components and a clear liquid supernatant. The supernatant was then further refined by lyophilization and reconstitution in 10% of the original liquid volume to drive the PBS concentration to 10× (1.4 M NaCl, 27 mM KCl, 0.119 M Phosphate), which salted out any remaining structural components. A quick spin to remove any debris yielded a concentrated solution containing the most soluble components of UBM hydrogel. Due to the limited yield of structural components during this salting out step, the material could not be recovered and was thus discarded. As evidence of the removal of structural proteins, the soluble fraction could no longer form a hydrogel. In contrast, the gel pellet—containing the structural components—was completely insoluble at neutral pH and had to be homogenized by repeated pipetting for use in cell culture assays. Both the structural and soluble components were diluted to the original sample volume and the protein content was approximated with the BCA assay. The parent material (UBM hydrogel) contained 2.9+0.2 mg/mL protein. The soluble fraction contained 0.8+0.2 mg/mL protein and the insoluble scaffold contained 1.7+0.1 mg/mL protein indicating that ˜85% of the protein content was retained during the fractionation process. The ˜15% reduction from the theoretical yield most likely resulted from the material discarded during the salting out step or the inaccuracy of estimating collagen rich protein samples in colorimetric protein assays. SDS PAGE analysis of the fractions shows that the structural fraction contained enriched quantities of high molecular weight (HMW) components (>80 kDa) compared to UBM hydrogel while the soluble fraction contained only very faint quantities of HMW proteins (FIG. 1B). The low molecular weight (LMW) end of the gel (<46 kDa) revealed that the soluble fraction contained significantly enriched quantities of LMW species compared to the other materials (FIG. 1C). The multi-banding pattern demonstrated by UBM hydrogel and the structural fraction is consistent with the protein signature of collagen. A picrosirius red stain of the structural fraction corroborated this result (FIG. 1D). Collectively, these results suggest this simple fractionation procedure effectively separated the insoluble, structural components of UBM hydrogel from the soluble components released during pepsin digestion.

### Effect of Fractions on PSCs

### Chemotaxis

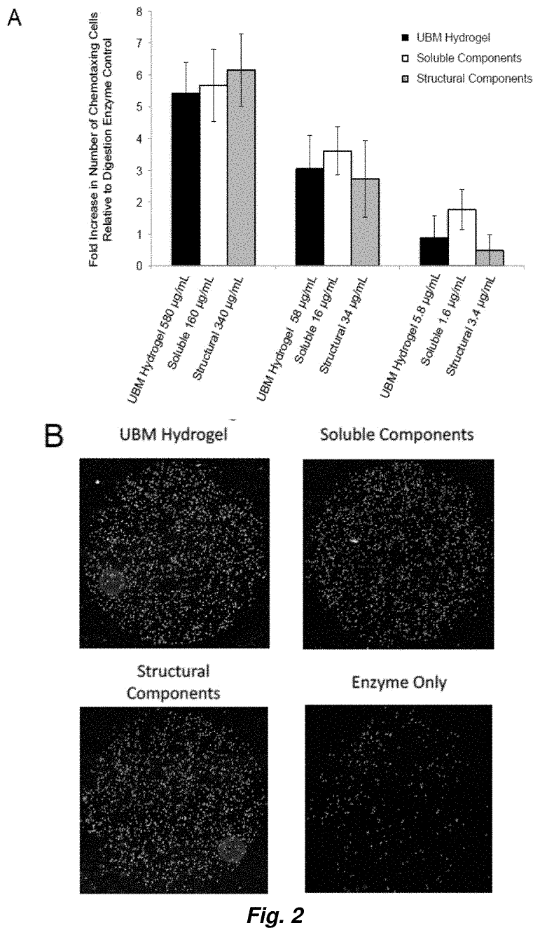

To determine the effect of crude fractionation on the chemotactic properties of UBM hydrogel, Boyden Chamber chemotaxis assays were carried out using PSCs. Previous studies have established that directed migration of a variety of different endogenous stem and progenitor cells towards implanted biologic scaffolds is a significant component of the remodeling response and PSCs have been well established as a model system for testing the chemotactic activity of ECM scaffolds in vitro. Stem cells were allowed to migrate through a polycarbonate filter towards UBM hydrogel and its soluble or structural components fractionated from UBM hydrogel for three hours before fixation and imaging. All of the materials were diluted five fold from their stock solutions yielding final concentrations of 580 μg/mL UBM, 160 μg/mL soluble components, and 340 μg/mL structural components. Strikingly, both fractions retained roughly equivalent chemotactic activity to the parent material over three orders of magnitude in concentration (FIG. 2). The increase in cellular chemotaxis ranged from a 5-6 fold increase over digestion enzyme control at the highest concentration tested to a 0.5-2 fold increase at the lowest concentration (FIG. 2). No significant differences between materials were observed at any of the concentrations tested. The chemotactic activity of the soluble fraction was particularly striking as this fraction contained significantly less protein than the other materials tested. Moreover, this result is consistent with a previous report of LMW peptides isolated from UBM having significant chemotactic activity [Agrawal V, Kelly J, Tottey S, Daly K A, Johnson S A, Siu B F, et al. An isolated cryptic peptide influences osteogenesis and bone remodeling in an adult mammalian model of digit amputation. Tissue Eng Part A 2011; 17:3033-44]. Collectively, the results presented here suggest that both the soluble and structural components of UBM hydrogel contribute to its chemotactic activity.

### Proliferation

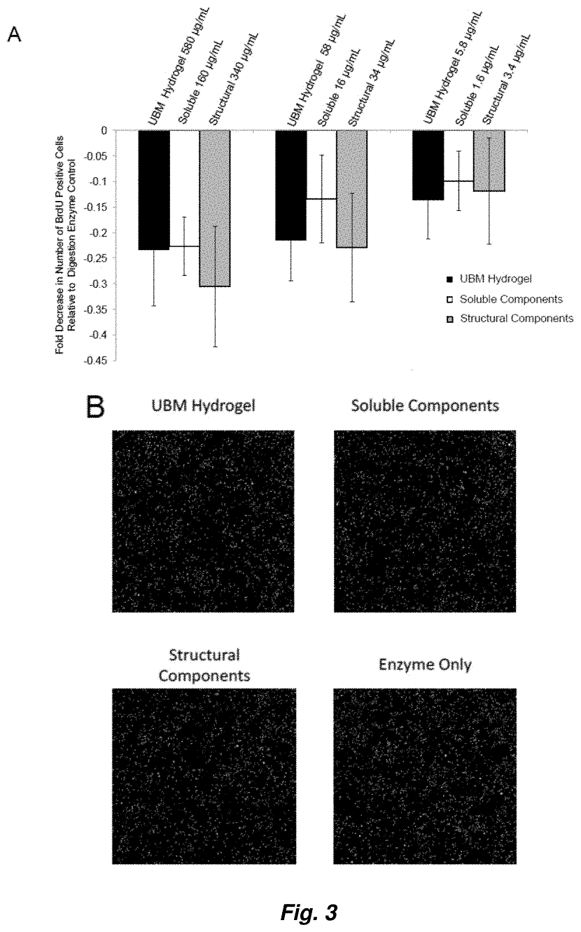

Biologic scaffolds comprised of ECM have been shown to affect the proliferation of stem cells, either positively or negatively, in a dose dependent and tissue source dependent fashion. To determine the effect of fractionation on proliferation of stem cells, PSCs were treated with UBM hydrogel and its fractionated soluble or structural components and were pulsed with BrdU for 18 hours. After incubation, the cells were stained, imaged, and the average number of BrdU+ DAPI+ nuclei for each condition was determined. All three materials decreased proliferation from control ranging from a 10% relative decrease at the lowest concentration tested to a 31% decrease at the highest concentration tested (FIG. 3). No significant differences were detected between UBM digest, and its soluble or structural components at any of the concentrations tested. These results suggest that both the soluble and structural molecules in UBM hydrogel contribute to the decrease in proliferation that is observed at high concentrations of ECM.

### Effect of Fractions on Macrophage Behavior

### Phagocytosis

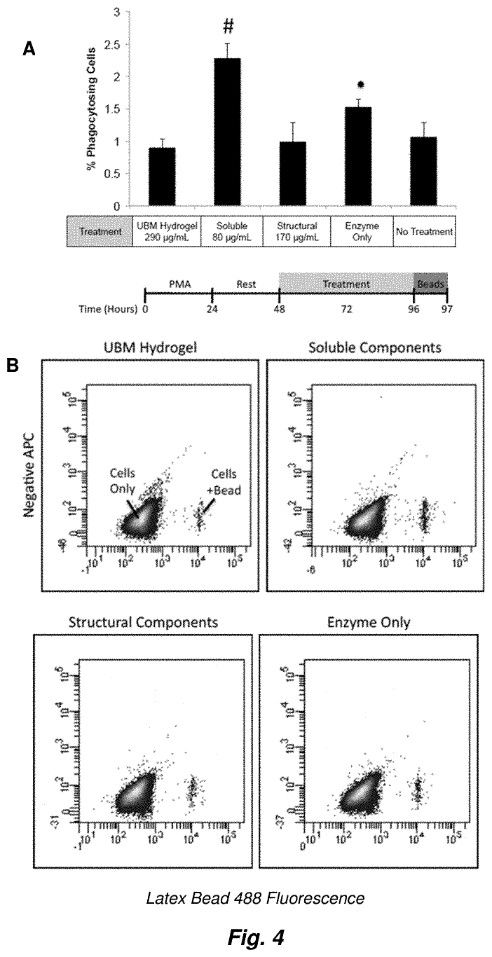

One of the primary functions of macrophages during tissue remodeling is the phagocytosis of invading pathogens and debris. Given that macrophages are indispensible for ECM mediated constructive remodeling, the effects of UBM hydrogel and its structural or soluble components on the phagocytic ability of THP1 human macrophages were determined. THP1 macrophages were treated with UBM and its structural or soluble components for 48 hours after which their ability to phagocytose a fluorescent latex particle was examined Quantification of phagocytosis via flow cytometry revealed that macrophages treated with soluble components of UBM hydrogel increased phagocytosis 1.2 fold from control. Neither the structural components nor the UBM hydrogel itself had any effects on macrophage phagocytosis (FIG. 4A).

### Cytokine Secretion

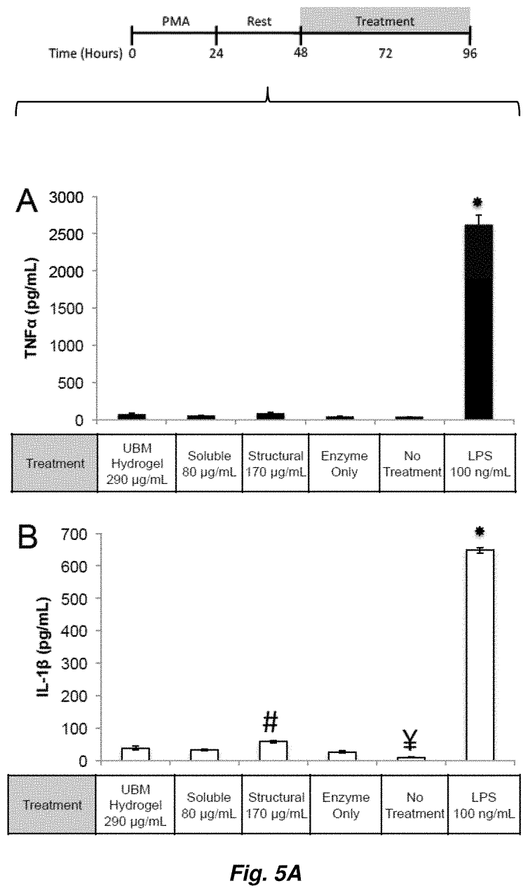

An additional function of macrophages in tissue remodeling is the propagation or minimization of inflammation via the production and secretion of soluble factors. Studies have shown that these events are tightly regulated both spatially and temporally, and inhibiting these events can lead to poor remodeling outcomes. To examine the role of soluble and structural components of UBM hydrogel in modulating macrophage inflammation, TNFα and IL-1β secretion from THP-1 macrophages, treated with UBM hydrogel, its structural, or its soluble components were quantified. Treatment with these materials for 48 hours slightly increased production of these cytokines above control (1-2 fold, FIG. 5A,B). However, in comparison to pro-inflammatory stimuli (LPS, 100-1000 fold above control) these increases were quite minor and could be attributable to small quantities of endotoxin in the source tissue or pepsin enzyme. Given that neither of the fractionated components yielded a robust inflammatory response, we sought to characterize their ability to prevent inflammation. Previous studies have demonstrated that M2 cytokines such as IL-4 and IL-10 can prevent secretion of TNFα and IL-1β when pre-administered or co-administered with M1 cytokines (LPS and/or IFNγ) and this response may be important for macrophage immunomodulation during tissue remodeling. To determine if UBM hydrogel or its fractionated components could prevent inflammation, macrophages were pretreated with these materials for 48 hours and challenged by treatment with LPS for 2 hours (FIG. 5C,D).

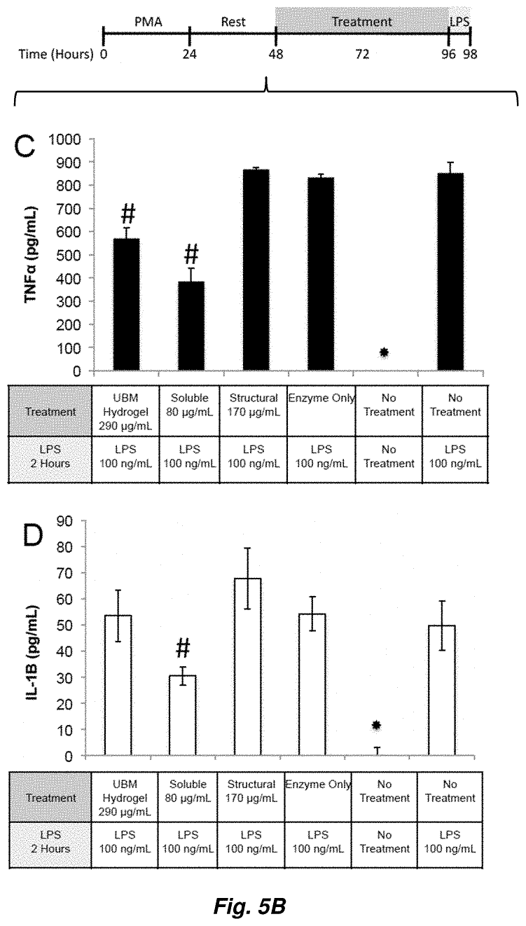

Pretreatment of macrophages with UBM and its soluble components significantly prevented TNFα secretion with 35% and 60% reductions from untreated control respectively (FIG. 5C). However, the structural components of UBM digest did not prevent TNFα secretion. Soluble components also prevented IL-1β secretion by 33% while UBM hydrogel and structural components did not prevent IL-1β secretion relative to untreated control (FIG. 5D). One possible mechanism for this anti-inflammatory activity is the upregulation and secretion of interleukin receptor inhibitors such as the IL-1 receptor antagonist (IL1-RA). To investigate whether this mechanism was occurring, macrophages were treated with UBM hydrogel, its soluble, and its structural components for 48 hours. Treatment with the soluble components increased IL-1RA production 2.4 fold from enzyme only control while UBM and its structural components only induced 0.3 fold increases (FIG. 6). Collectively, these results suggest that the soluble components of UBM hydrogel play an important role in macrophage immunomodulation.

### Prostaglandin Secretion

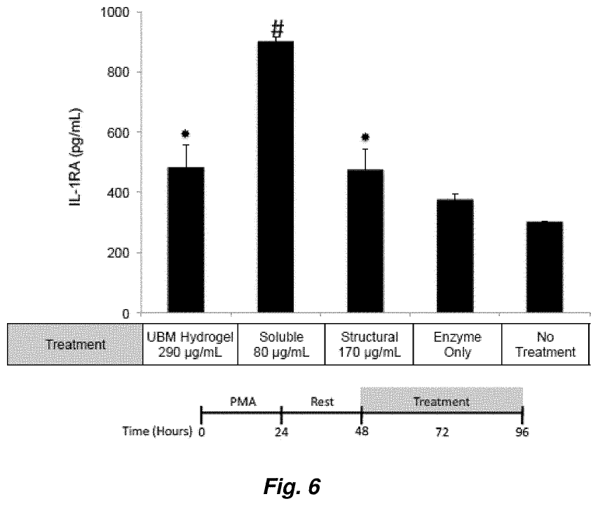

Macrophages help orchestrate tissue remodeling by releasing a variety of proteins and small molecules. One well-studied class of small molecules that macrophages utilize in this capacity are the prostaglandins. Upregulation of COX2 by macrophages to drive prostaglandin production has been shown to alter cell proliferation, phagocytic behavior, and new matrix deposition. Thus the role of structural and soluble components in modulating prostaglandin release from macrophages, structural and soluble components of UBM hydrogel were exposed to macrophages for 72 hours. Treatment of THP1 macrophages with UBM and its structural components increased PGE2 production from undetectable quantities up to 620 and 810 pg/mL, respectively. In contrast, the soluble components of UBM digest did not increase PGE2 above untreated levels (FIG. 7A). A similar observation was made for PGF2α as UBM digest and its structural components nearly doubled the quantity of secreted PGF2α while the soluble components did not cause any significant changes in PGF2α concentration (FIG. 7B). To confirm the role of COX2 in this assay, macrophages were co-incubated with the COX2 inhibitor, NS-398 (FIG. 7C). Addition of NS-398 abrogated PGE2 secretion down to control levels. These results suggest that the structural components of UBM digest also contribute to the remodeling response but in a different capacity than the soluble components.

### In Vivo Remodeling

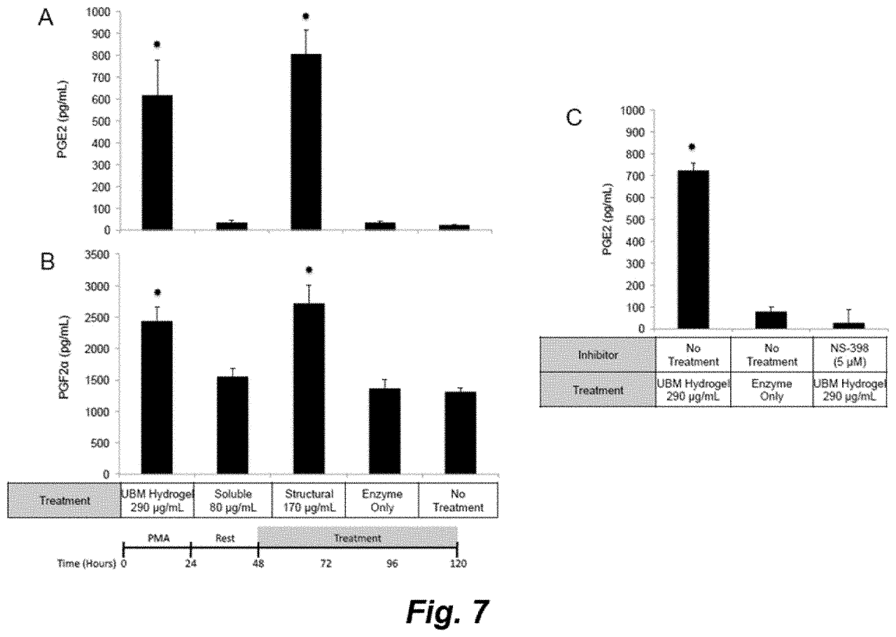

Previous animal studies have examined the phenotype of macrophages infiltrating both UBM sheets and hydrogels and determined that a prominent CD206+ macrophage response is predictive of a constructive remodeling outcome. However, the prostaglandin response, which originates from an increase in COX2 expression, has not been investigated. Thus the expression of COX2 by macrophages invading UBM hydrogels in vivo was determined. Tissue sections from rats implanted with UBM hydrogel into an abdominal wall defect were survived for 3 and 7 days. These sections were colabeled for both CD68 (pan-macrophage) and COX2 expression. A dense cell infiltrate into the gel with a robust macrophage (CD68+) was observed. Interestingly, the majority of cells expressing CD68 also expressed COX2 at both 3 days and 7 days post-implantation (FIG. 8A). To determine if COX2 might also be associated with a constructive remodeling outcome, the aforementioned tissue sections were colabeled for CD206 and COX2. Strikingly, at both 3 and 7 days, an overwhelming majority of cells expressing CD206 also expressed COX2. These results suggest that during the acute phase of remodeling, COX2 may indeed be a component of the UBM hydrogel initiated constructive remodeling response.

### Discussion

While significant progress has been made in the development and implementation of ECM based hydrogels, very little is known about the molecular components of these materials that facilitate the tissue remodeling response. The data presented herein suggest that both the structural components and the soluble components of the hydrogel contribute to the tissue remodeling response by stimulating stem cell chemotaxis, a decrease in stem cell proliferation, and the development of an anti-inflammatory, pro-remodeling response in macrophages. While chemotaxis and proliferation are common features of both the soluble and structural components of the hydrogel, macrophage behavior is differentially regulated with structural components contributing to the production of prostaglandins and soluble components contributing to a suppression of classic inflammation as well as an increase in phagocytosis.

The equivalent biological activity of the structural and soluble components in driving PSC chemotaxis and proliferation is consistent with the currently proposed paradigm of ECM scaffold driven constructive remodeling. It is well accepted that both matricryptic peptides released from full-length structural ECM proteins during proteolysis as well as matricryptic sites unmasked on the scaffold itself during proteolysis, can drive cellular chemotaxis and proliferation as well as other behaviors such as cell shape and adhesion. Additionally, full-length growth factors, which can be freely soluble or tightly bound to structural components of ECM, are capable of driving stem cell chemotaxis and proliferation. In the context of ECM hydrogel driven tissue remodeling, the biologic activity in both fractions could help control both the intensity and the duration of the host response. Studies on hydrogels have shown that embedded soluble factors diffuse from hydrogels in a controlled fashion that is governed by a variety of hydrogel properties. In remodeling ECM scaffolds, the soluble components of the hydrogel could diffuse into nearby tissues providing a prolonged burst to drive stem cell recruitment and proliferation. Subsequent to the initial burst, the structural components could provide a longer-term chemotactic and proliferative signal to continue driving the host response. This controlled release could help explain why ECM hydrogels provide a more robust remodeling response than collagen hydrogels alone that may not contain the diversity and quantity of soluble components.

The immunomodulatory effects of the structural and soluble components on macrophages support many of the currently accepted models for tissue remodeling. As aforementioned, a freshly implanted hydrogel would likely provide a burst of soluble components into the surrounding tissue. Given the results of this study, those soluble components could work to suppress the classic inflammatory response while increasing the phagocytic activity of macrophages as they migrate towards the hydrogel. Upon arrival and interaction with structural components, these macrophages would begin upregulating COX2 in a localized fashion. Both the spatial location and controlled release of these events could be important for scaffold and tissue remodeling. Studies on tumor-associated macrophages (TAMs) have shown that newly infiltrating macrophages acquire their phenotype (M1 or M2) as they move up a gradient of growth factors released by TAMs. The release of soluble components from the hydrogel could prime infiltrating macrophages with a burst of phagocytic activity to clean up the wound site and prevent sepsis while also driving them to an anti-inflammatory phenotype. Once those macrophages arrive at the wound site and interact with the hydrogel, the localized upregulation of COX2 and prostaglandins could begin to orchestrate different facets of the remodeling response including collagen synthesis, matrixmetalloproteinase (MMP) release, stem cell proliferation, stem cell differentiation, and neovascularization. The localization of COX2 upregulation could also be highly important. Many disease models involving inflammation and hyperalgesia purport that low levels of prostaglandins are beneficial while high levels are deleterious. Localizing COX2 upregulation to the anatomic site of the hydrogel likely limits exposure while also providing the necessary burst of prostaglandins for constructive remodeling. This localized behavior is also consistent with previous studies, which have shown that cell shape, growth factor potency, and gene regulation are modulated or enhanced if cells are in contact with structural ECM components (collagen, fibronectin, laminin, etc.).

The data presented herein also emphasize the plasticity of macrophages. The secretion profile exhibited by macrophages in this study is consistent with that of M2, regulatory macrophages. While M2 macrophages are well documented in ECM mediated constructive remodeling, the differential secretion profiles induced by soluble and structural components in this study corroborates a model whereby ECM remodeling involves several sub-phenotypes (M2a, M2b, M2c) of M2 macrophages working in concert and adjusting their sub-phenotype in response to environmental cues. In addition, these studies underscore the importance of analyzing a variety of surface and secretory markers to describe macrophage phenotype rather than restricting analysis to one or two markers. Further studies are required to identify a minimum subset of markers to quantitatively describe the macrophage population during ECM mediated constructive remodeling. Determination of this subset may provide a framework to enhance the constructive remodeling response to ECM hydrogels by augmenting macrophage phenotype and function.

### 5. Conclusions

The structural and soluble components of ECM hydrogels contribute to the improved tissue remodeling outcomes facilitated by these materials. Both components promote chemotaxis and changes in proliferation of stem cells as well as changes in macrophage behavior. In the context of macrophage behavior, these materials initiate distinctive cellular responses with structural components driving a COX2 upregulation and soluble components promoting phagocytosis and suppressing inflammation. These studies provide a framework for a more detailed characterization of the immunomodulatory effect of ECM hydrogels.

### Example 2

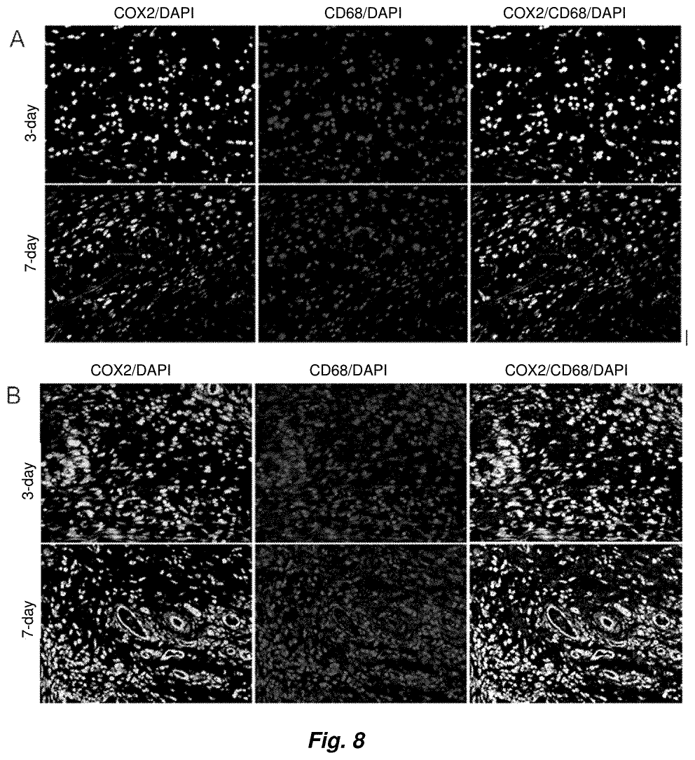

As indicated above, it was demonstrated that the structural components mediated prostaglandin secretion in THP1 macrophage-like cells that was independent of a classical inflammatory response (TNFα, IL-1β, etc.). We expanded upon these results by showing that if we more minimally digest our UBM with pepsin to preserve structural components, we see an increased output of prostaglandins (FIG. 9).

### Example 3

The findings above also were expanded upon by targeting the hyaluronic acid component in UBM for degradation using the enzyme hyaluronidase. Digestion of the hyaluronan component in UBM completely abrogated the prostaglandin response. An image of the data is shown in FIG. 10. This data further confirms the bioactivity of a structural fraction of UBM and also suggests a possible molecular origin.

### Example 4

Here we show that inhibiting the bioactivity of the structural fraction using a COX1/2 inhibitor (Aspirin) reduces the therapeutic benefit of the materials. Treatment of an abdominal muscle injury in rats with UBM leads to a pro-myogenic response along with a robust deposition of collagen. Inhibiting the structural component response of UBM with Aspirin reduces total collagen deposition as well as myogenesis. These results are confirmed with in vitro tests.

Biologic scaffolds composed of ECM have been widely used to reinforce the surgical repair of soft tissue defects and to mediate an improved or constructive remodeling outcome. While the clinical applications of ECM scaffolds are quite diverse and constantly expanding, skeletal muscle reinforcement (e.g. hernia repair and volumetric muscle loss) remains one of the most prevalent clinical applications for these scaffolds. When placed at the site of injury, ECM scaffolds orchestrate a complex host response that includes the recruitment of endogenous cells, such as immune cells and stem/progenitor cells. Degradation of the scaffold by infiltrating host cells releases a variety of bioactive molecules that drive neovascularization, innervation, and site appropriate tissue formation.

One important feature of ECM scaffolds during the remodeling process is their ability to modulate macrophage phenotype. ECM scaffolds from a variety of source tissues promote an M2-like bias (CD163^{high}, CD206^{high}, CD86^{low}, CCR7^{low}) in the infiltrating macrophage population. This bias has been shown to be a determinant factor in a favorable tissue remodeling outcome. While a complete characterization of macrophage phenotype during tissue remodeling has yet to be completed, several studies have begun to describe this M2-like phenotype.

As shown above, an enzymatically digested ECM scaffold derived from porcine urinary bladder (urinary bladder matrix, UBM) was found to up-regulate prostaglandin-E2 (PGE2) and prostaglandin-F2α (PGF2α) secretion in macrophages as part of a larger change in the overall macrophage phenotype. Prostaglandin production requires the cyclooxygenase enzymes COX1 (constitutively expressed) and COX2 (inducibly expressed). Several studies have shown that COX2 knockout macrophages do not become fully M2 polarized and assume an M1-like phenotype. Moreover, while prostaglandins can enhance the inflammatory response and pain states, these molecules are important mediators of tissue repair particularly in the context of skeletal muscle. Collectively, these observations imply a potentially important role for COX1/2 in ECM-mediated macrophage polarization, and ultimately in constructive remodeling of ECM scaffolds.

COX1/2 inhibitors such as NSAIDs are routinely administered post-surgically, primarily for anti-inflammatory and analgesic purposes. While COX1/2 inhibitors are important pain management, they have also been shown to delay or diminish the healing process, including macrophage accumulation; leading some to question their clinical use in treating musculotendinous injuries. To date, no study has been conducted to determine if the administration of NSAIDs would similarly affect ECM scaffold remodeling. The purpose of the following was to determine the effect of a common NSAID, Aspirin, on the constructive remodeling response—including macrophage phenotype—mediated by a clinically relevant ECM scaffold, UBM, in a rat skeletal muscle injury model.

### Materials and Methods

### Overview of Experimental Design

An established rodent skeletal muscle injury model was used to evaluate the effect of the COX1/2 inhibitor, Aspirin, on the ECM scaffold mediated constructive remodeling response. Briefly, three days prior to the surgical procedure, animals were randomly assigned to either the Aspirin treated (3 mg/mL Aspirin in drinking water) or control (vehicle) group. Bilateral 1.5 cm×1.5 cm partial thickness defects were created in the abdominal musculature.Asize-matchedpre-cast UBM hydrogel and an overlying 2×2 cm single layer sheet of UBM was then placed in the muscle defect area. The remodeling response was evaluated following 3, 7, 14, and 35 days by quantitative histomorphologic metrics (Wolf M T, Carruthers C A, Dearth C L, Crapo P M, Huber A, O A, Londono R, Johnson S A, Daly K A, Stahl E C and others. Polypropylene surgical mesh coated with extracellular matrix mitigates the host foreign body response. J Biomed Mater Res A 2013 and Wolf M T, Daly K A, Brennan-Pierce E P, Johnson S A, Carruthers C A, D'Amore A, Nagarkar S P, Velankar S S, Badylak S F. A hydrogel derived from decellularized dermal extracellular matrix. Biomaterials 2012; 33 (29): 7028-38), including characterization of macrophage phenotype and neo tissue deposition.

Established in vitro models were subsequently used to further interrogate the effect of Aspirin on ECM scaffold mediated macrophage function/polarization and myogenesis. In vitro macrophage function and polarization was characterized by quantification of secreted factor production and cell surface marker expression, respectively. In vitro myogenesis was characterized by an objective image analysis system which quantified fusion index, an important myogenesis parameter.

### Reagents

All chemicals were purchased from Sigma-Aldrich (St. Louis, Mo.) unless otherwise specified. All cell culture supplies were purchased from Life Technologies (Carlsbad, Calif.) unless otherwise specified. All chemicals used in this study were molecular biology grade or cell culture grade where appropriate.

### Urinary Bladder Matrix Preparation

Porcine urinary bladders were acquired from Tissue Source, LLC. (Lafayette, Ind.). The ECM prepared from this tissue and referred to as UBM was prepared as previously described (Freytes D O, Tullius R S, Badylak S F. Effect of storage upon material properties of lyophilized porcine extracellular matrix derived from the urinary bladder. J Biomed Mater Res B Appl Biomater 2006; 78(2):327-33). Briefly, the tunica serosa, tunica muscularis externa, tunica submucosa, and tunica muscularis mucosa were mechanically removed. The luminal urothelial cells of the tunica mucosa were dissociated by washing with sterile water. The remaining tissue consisting of basement membrane, subjacent tunica propria of the tunica mucosa, and any resident cells in those layers was decellularized by agitation in 0.1% peracetic acid with 4% ethanol for 2 hours at 300 rpm. The tissue was then extensively rinsed with phosphate-buffered saline (PBS) and sterile water. The UBM was then lyophilized into a dry sheet and used as is, where appropriate or milled into particulates using a Wiley Mill with a #60 mesh screen (Gilbert T W, Stolz D B, Biancaniello F, Simmons-Byrd A, Badylak S F. Production and characterization of ECM powder: implications for tissue engineering applications. Biomaterials 2005; 26 (12):1431-5).

### Pepsin Mediated ECM Solubilization and Hydrogel Formation

UBM was enzymatically digested with pepsin as described (Freytes D O, Martin J, Velankar S S, Lee A S, Badylak S F. Preparation and rheological characterization of a gel form of the porcine urinary bladder matrix. Biomaterials 2008; 29(11):1630-7). Briefly, milled UBM particulates (10 mg/mL) and pepsin (1 mg/mL) were placed in 0.01 M HCl (pH 2.0, sterile filtered) and stirred at room temperature for 48 hours. The thick slurry was then neutralized to a pH of 7.4 in sterile 1×PBS (137 mM NaCl, 2.7 mM KCl, 12 mM Phosphate, Fisher Scientific, Waltham, Mass.) to inactivate the pepsin. A solution of pepsin (1 mg/mL) in 0.01M HCl, treated in the same fashion as the UBM sample, served as the control condition for all experiments. All steps were conducted under sterile conditions with sterile filtered solutions. To form hydrogels, the neutralized slurry was placed in a 1.4×1.4×0.5 cm plastic mold and incubated at 37° C. for 30 minutes. For cell culture experiments the solid UBM hydrogel was broken into smaller pieces with vigorous agitation and pipetting. The subsequent slurry was then added directly to cells. For animal studies the UBM hydrogel was removed from the mold and placed directly into the defect site.

### In-Vivo Study

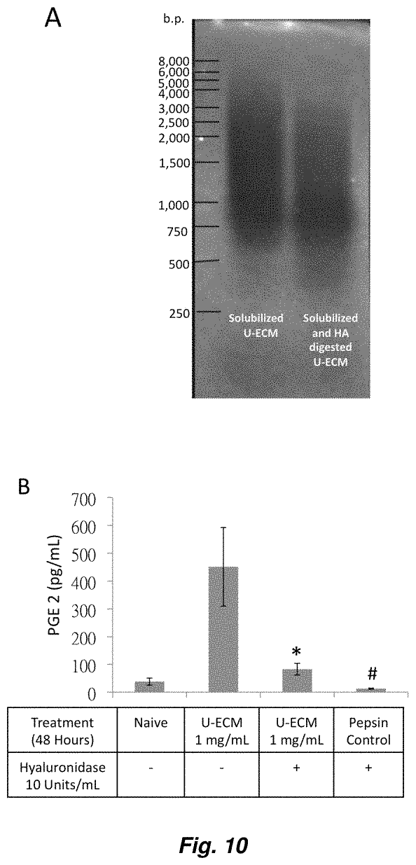

Thirty-two female Sprague Dawley rats (350-400 g at implantation) were purchased from Harlan Laboratories. Rats were housed on a 12 hour light-dark cycle and fed standard laboratory chow and water ad libitum. Animals were randomly assigned to either the Aspirin treated or control (vehicle) group. Three days prior to surgery, animals in the Aspirin group had their drinking water supplemented with 3 mg/mL Aspirin which was continued throughout the experimental time course. Consumption of water and animal weight was tracked daily throughout the duration of the study. Salicylates in whole blood were determined using the Salicylates Detection Kit from Neogen according to the manufacturer's instructions. Analysis of circulating salicylate content revealed a total salicylate concentration of 64 μg/mL in the NSAID treated group and no detectable salicylate content in the untreated group (FIG. 11).

### Surgical Procedure

Anesthesia was induced with 2.5-4% isoflurane inhalant anesthetic and maintained at 0.5-3% throughout the procedure. The ventral abdomen was prepared for aseptic survival surgery by clipping the fur over the entire abdominal region, and cleaning the operative area with three alternating scrubs of providone-iodine surgical scrub and 70% isopropyl alcohol solutions. A final preparation of 70% isopropyl alcohol was applied and allowed to dry, followed by an application of DuraPrep™, which was allowed to dry before applying and placing sterile surgical drape(s) over the entire field.

A 4 cm midline skin incision was made and the skin was bluntly reflected to expose the abdominal muscle. Bilateral 1.5 cm×1.5 cm partial thickness paramedian defects approximately 1 cm apart were created in the abdominal muscle. The defects were filled with size-matched pre-cast UBM hydrogels placed in the defect area (n=4 implants/group/time point). To prevent migration of the hydrogel, a 2×2 cm single layer sheet of UBM was placed over the hydrogel and secured with 4-0 PROLENE™ interrupted sutures placed at the corners. The skin was closed with a continuous (inner) 4-0 VICRYL™ suture. Upon completion of the surgical procedure, the inhalant anesthetic was discontinued, and the animal was allowed to recover from anesthesia. The animal was given access to food and water ad libitum. Daily observations of each animal were made. The abdominal region of each animal was examined to assess both the condition of the wound line and subcutaneous tissues (e.g., dehiscence, seromas and/or hematomas).

### Test Article Harvest

Euthanasia was administered by CO₂ inhalation and subsequent cervical dislocation, which was performed in accordance with the American Veterinary Medical Association (AVMA) Guidelines on Euthanasia. Following euthanasia, the skin was gently dissected, reflected, and photographs were taken of each defect in situ. After completion of the initial examination, the entire body wall that includes the test article was explanted en bloc. The specimen was then cut in half and each half immersed in 10% Neutral Buffered Formalin (NBF) for histologic analysis.

### Immunolabeling and Quantification

CD206 and CD86: Cellular expression of markers of macrophage polarization (CD206 and CD86) were determined by immunolabeling using previously described methods (Keane T J, Londono R, Carey R M, Carruthers C A, Reing J E, Dearth C L, D'Amore A, Medberry C J, Badylak S F. Preparation and characterization of a biologic scaffold from esophageal mucosa. Biomaterials 2013; 34(28):6729-37). Briefly, paraffin embedded sections were washed with xylene and rehydrated slowly with an ethanol gradient. Antigen retrieval was accomplished by heating the sections to 95° C. in 0.01 M citrate buffer (pH=6) for 25 min. Tissue sections were washed in Tris-Buffered Saline Tween-20 (TBST) and incubated in blocking buffer consisting of 2% horse serum albumin, 1% BSA, 0.05% Tween-20, and 0.05% Triton X-100 in TBS for 1 hour. For M1/M2 phenotype analysis, the primary antibodies, mouse anti-rat CD68 (pan-macrophage marker, 1:100 AbD Serotec, Raleigh, N.C.), goat anti-rat CD206 (M2 marker, 1:100 Santa Cruz, Dallas, Tex.), and rabbit anti-rat CD86 (M1 marker, 1:250, Abcam, Cambridge, England) were diluted in blocking buffer, added to the sections and incubated overnight at 4° C. in a humidified chamber. After primary labeling, tissue sections were washed extensively in PBS and species appropriate secondary antibodies (donkey anti-rabbit Alexa-Fluor 488 (1:300), donkey anti-mouse Alex-Fluor 594(1:300), donkey anti-goat PerCP Cy5.5 (1:300), Life Technologies, Carlsbad, Calif.)) were added and incubated for 1 hour at room temperature in a humidified chamber. Tissue sections were further washed with TBST and imaged by a blinded observer at 40× magnification using a Nikon Eclipse E600 microscope (Chiyoda, Tokyo) with CRi Nuance FX multispectral imaging system (Cambridge, Mass.). Four representative images of each colabeled tissue section were collected. The number of CD68±CD206+ (M2-like macrophages) and CD68±CD86+ (M1-like macrophages) cells per field of view (40× magnification) were quantified using a custom Cell Profiler pipeline (Carpenter A E, Jones T R, Lamprecht M R, Clarke C, Kang I H, Friman O, Guertin D A, Chang J H, Lindquist R A, Moffat J and others. CellProfiler: image analysis software for identifying and quantifying cell phnotypes. Genome Biol 2006; 7(10):R100).

Fast and Slow Myosin Heavy Chain:

Myosin heavy chain positive cells in the defect area were determined by immunolabeling for fast and slow myosin heavy chain as previously described (Turner N J, Yates A J, Jr., Weber D J, Qureshi I R, Stolz D B, Gilbert T W, Badylak S F. Xenogeneic extracellular matrix as an inductive scaffold for regeneration of a functioning musculotendinous junction. Tissue Eng Part A 2010; 16(11):3309-17; Valentin J E, Turner N J, Gilbert T W, Badylak S F. Functional skeletal muscle formation with a biologic scaffold. Biomaterials 2010; 31(29):7475-84; and Wolf M T, Daly K A, Reing J E, Badylak S F. Biologic scaffold composed of skeletal muscle extracellular matrix. Biomaterials 2012; 33(10):2916-25). Briefly, slides were deparaffinized before epitope retrieval in 0.1 mM EDTA at 95° C. for 25 min followed by 0.1% trypsin/0.1% calcium chloride (w/v) at 37° C. for 10 min Peroxidase activity in tissue sections was quenched by incubation in 0.3% (v/v) hydrogen peroxide solution in TBS for 10 min Sections were then blocked with 2% horse serum, 1% BSA in TBS for 30 min Sections were then immunolabeled for mouse anti-slow myosin heavy chain (1:1000, clone NOQ7.5.4D, M8421, SigmaAldrich) for 40 min and subsequently rinsed in TBS. Sections were incubated in biotinylated goat anti-mouse IgG secondary antibody (1:200, Vector) for 1 h at room temperature and rinsed in TBS. Sections were then stained in Vectastain ABC reagent (Vectastain Elite ABC Kit, Vector) for 30 min and developed with a diaminobenzadine substrate (ImmPact DAB, Vector). Sections were incubated in blocking solution for 10 min before incubation in alkaline phosphatase conjugated mouse anti-fast myosin heavy chain (1:200, clone MY-32, A4335, Sigma) for 1 hour. Color was developed by staining with alkaline phosphatase (Red Alkaline Phosphatase Kit, SK-5100, Vector), dehydrated, and mounted for imaging by blinded observers. To quantify the effect of Aspirin on ECM scaffold induced myogenesis, the myogenic index (total cross sectional area of myosin heavy chain positive cells as a function of the total defect area) was quantified at the 35 day timepoint. Specifically, mosaic images spanning the entire defect were obtained using a Zeiss Axio-Observer Z.1 microscope (Oberkochen, Germany), and each myosin heavy chain positive cell border was traced and the area quantified with ImageJ software. A blinded observer distinguished the location of the defect border from the intact native tissue and identified myogenesis by the presence of centrally located nuclei within cells that were also positive for myosin heavy chain. The MHC area at the earlier time points was not determined as a previous study showed that no myogenesis with UBM is observed before 35 days (Wolf M T, Daly K A, Brennan-Pierce E P, Johnson S A, Carruthers C A, D'Amore A, Nagarkar S P, Velankar S S, Badylak S F. A hydrogel derived from decellularized dermal extracellular matrix. Biomaterials 2012; 33(29):7028-38).

Picrosirius Red Staining and Imaging.

The area of collagen fibers as a function of their color hue was quantified from tissue sections stained with picrosirius red and imaged with circularly polarized light microscopy at 20× magnification. The color hue corresponds to relative fiber thickness from thin green fibers to increasingly thick yellow, orange, and red fibers (Wolf M T, Carruthers C A, Dearth C L, Crapo P M, Huber A, Burnsed O A, Londono R, Johnson S A, Daly K A, Stahl E C and others. Polypropylene surgical mesh coated with extracellular matrix mitigates the host foreign body response. J Biomed Mater Res A 2013; Cuttle L, Nataatmadja M, Fraser J F, Kempf M, Kimble R M, Hayes M T. Collagen in the scarless fetal skin wound: detection with picrosirius-polarization. Wound Repair Regen 2005; 13(2): 198-204; and Nadkarni S K, Pierce M C, Park B H, de Boer J F, Whittaker P, Bouma B E, Bressner J E, Halpern E, Houser S L, Tearney G J. Measurement of collagen and smooth muscle cell content in atherosclerotic plaques using polarization-sensitive optical coherence tomography. J Am Coll Cardiol 2007; 49(13):1474-81). A custom Matlab (The Mathworks, Natick, Mass.) script transformed each image from the RGB to the HSV color model, separated each color component as a function of hue (red 2-9 and 230-256, orange 10-38, yellow 39-51, green 52-128), applied a threshold to remove noise from an average of a global threshold using Otsu's method (intensity value of 50/256), and expressed the collagen content for each color component as a percentage of the area of each image.

### Macrophage Cell Culture

THP-1 human monocytes were obtained from the American Tissue Culture Collection (ATCC, Manassas, Va.) and maintained in RPMI, 10% FBS, 1% penicillin/streptomycin, 2 mM L-glutamine, and 1 mM sodium pyruvate in a humidified atmosphere at 37° C. with 5% CO2. For experiments, 500,000 THP-1 cells/mL were plated with 320 nM phorbol 12-myristate 13-acetate (PMA) for 24 hours to induce differentiation into macrophages. Adherent macrophages were washed in PBS and placed in fresh media, followed by a 24 hour incubation to acquiesce. Resting THP1 cells after differentiation has been shown to provide a macrophage-like cell with similar behavior to primary human peripheral blood mononuclear cells (Daigneault M, Preston J A, Marriott H M, Whyte M K, Dockrell D H. The identification of markers of macrophage differentiation in PMA-stimulated THP-1 cells and monocyte-derived macrophages. PLoS One 2010; 5(1):e8668).

Rat bone marrow mononuclear cells were matured to macrophages as previously described (Boltz-Nitulescu G, Wiltschke C, Holzinger C, Fellinger A, Scheiner O, Gessl A, Forster O. Differentiation of rat bone marrow cells into macrophages under the influence of mouse L929 cell supernatant. J Leukoc Biol 1987; 41(1): 83-91). Briefly, bone marrow cells were flushed from leg bones of female Sprague Dawley rats, triturated in isolation media (DMEM high glucose media with 2% Penicillin-Streptomycin) and centrifuged. The cell pellet was resuspended in red blood cell (RBC) lysis buffer (155 mM NH4Cl, 10 mM KHCO3, 0.9% EDTA in distilled water) and incubated for 15 minutes on ice. Cell debris was removed by centrifugation and the cell pellet was resuspended in macrophage maturation media (DMEM high glucose, 10% heat inactivated FBS, 20% L929 fibroblast conditioned media, 0.5 mM MEM non-essential amino acids, 2 mM L-glutamine, 10 mM Hepes pH 7.4, 1% penicillin/streptomycin, 0.12% 50 mM 2-mercaptoethanol). Cells were seeded at a density of 1 million/mL in 6 well plates and incubated at 37° C./5% CO2. Media was changed every 2-3 days for 7 days.

Matured bone marrow derived macrophages (BMDMs) were treated with 1 mL of StemPro® Accutase® (Invitrogen), incubated at 37° C. for 10 minutes, and detached with gentle pipetting. The cell suspension was collected and centrifuged for 5 mins at 1400 rpm. The cell pellet was resuspended in macrophage culture media (DMEM high glucose, 10% heat inactivated FBS, 0.5 mM MEM non-essential amino acids, 2 mM L-glutamine, 10 mM Hepes pH 7.4, 1% pennicillin/streptomycin, 0.12% 50 mM 2-mercaptoethanol) and plated at a density of 500,000/mL. Cells were allowed to recover overnight before treatment.

Primary microglia from rats were obtained from postnatal day 3 rat pup brains as previously described (Shaked I, Tchoresh D, Gersner R, Meiri G, Mordechai S, Xiao X, Hart R P, Schwartz M. Protective autoimmunity: interferon-gamma enables microglia to remove glutamate without evoking inflammatory mediators. J Neurochem 2005; 92(5):997-1009). Whole brains from postnatal day 3 Sprague Dawley rat pups (Charles River) were harvested and minced after removal of the brain stem, olfactory bulbs, and meninges. Tissue was digested for 15 mM in 0.25% trypsin/EDTA at 37° C. Brain tissues were triturated with a fire polished glass pipet in the presence of 1 mg/mL DNase I and 10% FBS. The suspension was centrifuged, diluted in culture media (DMEM/F12, 10% FBS, 2 mM L-glutamine, 1% penicillin/streptomycin), and grown to confluence at 37° C./5% CO2. Culture media was replaced every 2-3 days. At day 11-12 in vitro, enriched microglia were obtained by mechanical agitation (orbital shaker, 150 rpm, 37 C) for 2 hours. The media was collected after the shake-off step and centrifuged (1400 rpm for 5 minutes). The cell pellet was resuspended in culture media (RPMI 1640, 10% FBS, 1 mM L-glutamine, 1 mM sodium pyruvate, 50 μM beta-mercaptoethanol, 1% penicillin and streptomycin) and cells were plated at a density of 500,000/mL. Microglia were allowed to recover for 24 hours prior to treatment.

### Secreted Factor Analysis

Once cells were prepared as described above, aspirin (200 □M, where appropriate) was added for 1 hour. Subsequently, UBM hydrogel (0.5 mg/mL) or pepsin control buffer (0.05 mg/mL) was added and cells were allowed to incubate for 24-72 hours. After incubation, cells were pelleted by centrifugation (700×g, 5 minutes, 4° C.), and the culture supernatants were carefully removed. Supernatants were stored at −80° C. until the time of assay. Secreted factor concentrations in the culture supernatants were determined using commercially available ELISA kits (PGE2, PGF2α, ENZO Life Sciences) and IL-1RA (R&D Systems)) according to the manufacturer's instructions.

### SDS PAGE and Western Blotting

THP1 macrophage-like cells prepared as described in the cell culture section had 200 μM Aspirin added (where appropriate) 1 hour prior to UBM hydrogel (0.5 mg/mL) treatment. Cells were then treated with UBM hydrogel for 4, 8, or 24 hours. At the specified time points, cells were washed extensively with 1×PBS and lysis buffer (50 mM Tris, 20 mM NaCl, 1% Triton X-100) was added. Cells were mechanically removed from the culture dish, transferred to centrifuge tubes and incubated on ice for 10 minutes. Lysates were spun down at 14000 rpm for 20 minutes at 4° C. to remove any debris. The clarified lysate was stored at −80° C. Lysates were diluted 1:1 in 2× Laemmli Sample Buffer (Bio-Rad, Hercules, Calif.) and boiled at 95° C. for 5 minutes. Samples were then resolved on 9% SDS-PAGE gels (National Diagnostics) and transferred to polyvinyldifluoride (PVDF, Millipore) membranes. Membranes were blocked with 1:1 Odyssey Blocking Buffer:1×PBS (Licor) overnight at 4° C. Blocked membranes were immunoblotted with either rabbit anti-human COX2 (1:5,000, Abcam), goat anti-human CD206 (1:3,000, Santa-Cruz), rabbit anti-human CD86 (1:5,000, Abcam), or mouse anti-human β-Actin (1:5,000, Abcam) in blocking buffer for 1 hour at room temperature with agitation. Membranes were extensively washed with TBST before the appropriate secondary antibodies (1:10,000, donkey anti-goat IR Dye 680, donkey-anti rabbit IR Dye 680, and donkey anti-mouse IR Dye 800, Licor) in imaging buffer (1:1 Odyssey Blocking Buffer, 0.02% SDS in TBST) were added for 30 minutes with agitation. After extensive washing in TBST, membranes were imaging using the Licor Odyssey system. Densitometry was performed using ImageJ.

### In Vitro Myogenesis

C2C12 murine myoblasts were obtained from ATCC and grown in complete media (DMEM, 10% FBS, 1% penicillin/streptomycin) at 37° C./5% CO₂ according to the ATCC recommendations. Myoblasts were seeded at a density of 5,000 cells/cm² in 24 well plates and allowed proliferate to ˜90% confluence. Cells were then washed in 1×PBS and incubated in differentiation media (DMEM, 2% horse Serum, 1% penicillin/streptomycin) for 24 hours. Cells were washed in 1×PBS and placed in basal media (DMEM, 1% penicillin/streptomycin). A transwell insert (Corning) containing 100,000 differentiated THP1 cells (as described in section 2.9) in 200 μL of complete THP1 cell media was placed in each well. UBM hydrogel (0.5 mg/mL, final concentration) was then added to the THP1 transwell insert only.

Where appropriate, aspirin (200 μM) was added to the co-culture system first (i.e., ˜5 min before the UBM hydrogel). Pepsin control buffer (0.05 mg/mL) and non-treated cells served as the controls. The co-culture was incubated for 48 hours.

At the conclusion of the experimental duration the transwell inserts were removed and the C2C12 cells were washed in 1×PBS, fixed with 4% PFA for 15 minutes, permeabilized with 0.05% Triton X-100, treated with blocking buffer, and then immunolabeled with anti-sarcomeric myosin heavy chain (MHC) (1:20; clone MF20; Developmental Studies Hybridoma Bank) overnight at 4° C. Cells were then incubated with Alexa-Fluor 488 secondary antibody (1:400, Invitrogen) and mounted with Fluoromount-G™ containing 4′,6-Diamidino-2-phenylindole (DAPI; SouthernBiotech). A standardized image capture system and quantitative analysis of in vitro myogenesis was performed as previously described (Goh Q, Dearth C L, Corbett J T, Pierre P, Chadee D N, Pizza F X. Intercellular adhesion molecule-1 expression by skeletal muscle Cells Augments myogenesis. Exp Cell Res 2014). Briefly, custom macro functions (Image Pro 7; Media Cybernetics Inc.) were used to objectively quantify several important myogenesis parameters (e.g., the number of nuclei, myotubes, and nuclei within a myotube). Myotubes were operationally defined as MHC cells with 2 or more nuclei and an area greater than 200 μm². The fusion index was calculated by expressing the number of nuclei within myotubes as a percentage of total nuclei.

### Statistical Analysis

Where appropriate, a one-way or two-way analysis of variance (ANOVA) was performed to determine significant differences with Tukey post hoc testing (p<0.05). Data and error bars are reported as mean+standard deviation unless otherwise specified.

### Results

The purpose of the present study was to determine the effect of the COX1/2 inhibitor, Aspirin, on the ECM mediated constructive remodeling response, including macrophage phenotype and tissue deposition, in a rat model of skeletal muscle injury. Aspirin administration reduced myogenesis and collagen deposition in the remodeling area and was associated with a reduction in CD206 expressing M2-like macrophages and an increase in CD86 expressing M1-like macrophages. The effect of Aspirin on macrophage phenotype was further corroborated using an established in vitro model which showed augmented secreted factor production (PGE2, PGF2α) and cell surface marker expression (CD206, CD86).

### The Effect of Aspirin on Constructive Tissue Remodeling.

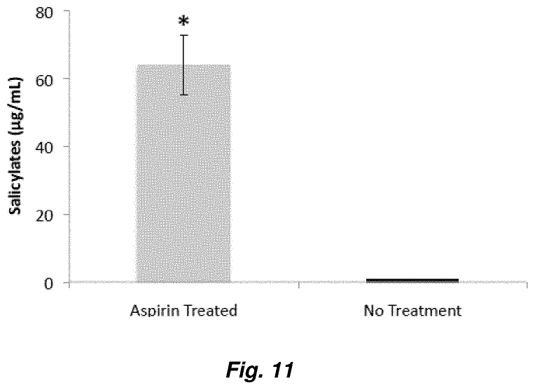

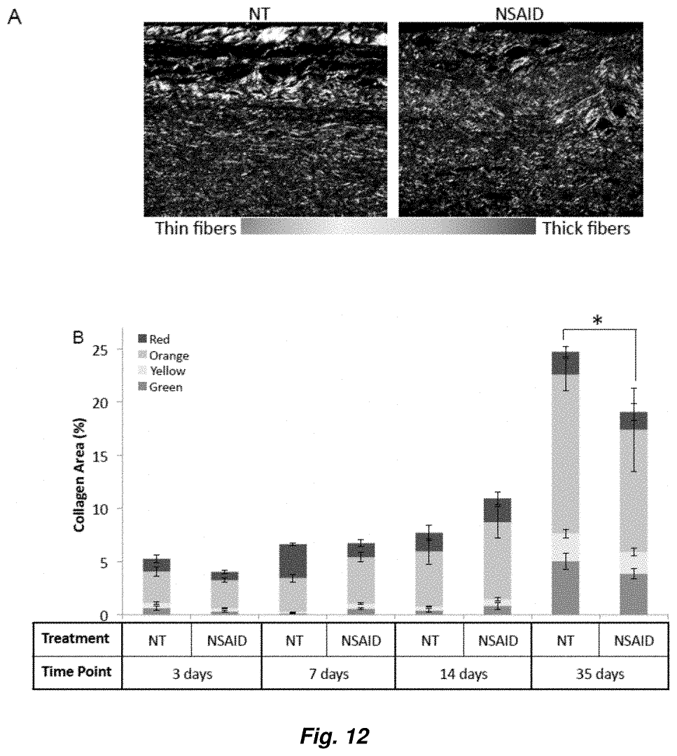

To quantify differences in tissue remodeling with Aspirin administration, the area of newly formed collagen and MHC cells within the defect borders were quantified using established metrics (Wolf M T, Carruthers C A, Dearth C L, Crapo P M, Huber A, Burnsed O A, Londono R, Johnson S A, Daly K A, Stahl E C and others. Polypropylene surgical mesh coated with extracellular matrix mitigates the host foreign body response. J Biomed Mater Res A 2013 and Wolf M T, Daly K A, Reing J E, Badylak S F. Biologic scaffold composed of skeletal muscle extracellular matrix. Biomaterials 2012; 33(10):2916-25). Picrosirius red staining of the defect area and imaging with polarized light microscopy showed a gradual increase in the abundance of collagen (5% to 25%) over the experimental time course (FIG. 12B). Administration of Aspirin altered the UBM-mediated remodeling response at the 35 day time point as shown by a 24% reduction in collagen deposition. ECM scaffold mediated de novo myogenesis was quantified by determining the myogenic index (FIG. 13A). At 35 days post implantation, a 4 fold reduction in myogenesis was observed in the Aspirin treated animals compared to non-treated controls (FIG. 13B). Collectively, these data suggest that Aspirin administration augments the UBM mediated constructive remodeling response by reducing both collagen content and myogenesis in the defect area.

### The Effect of NSAIDS on Macrophage Phenotype In Vivo.

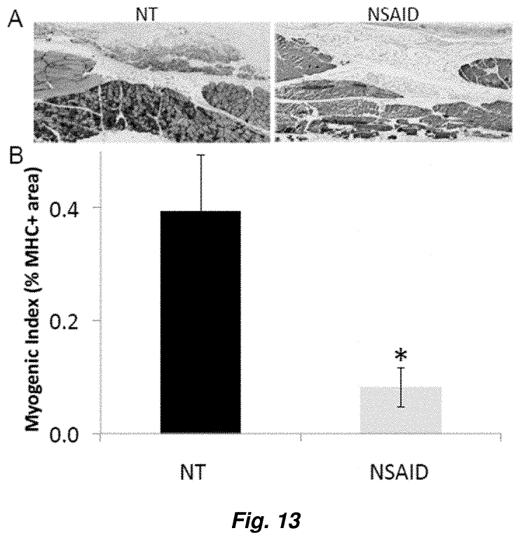

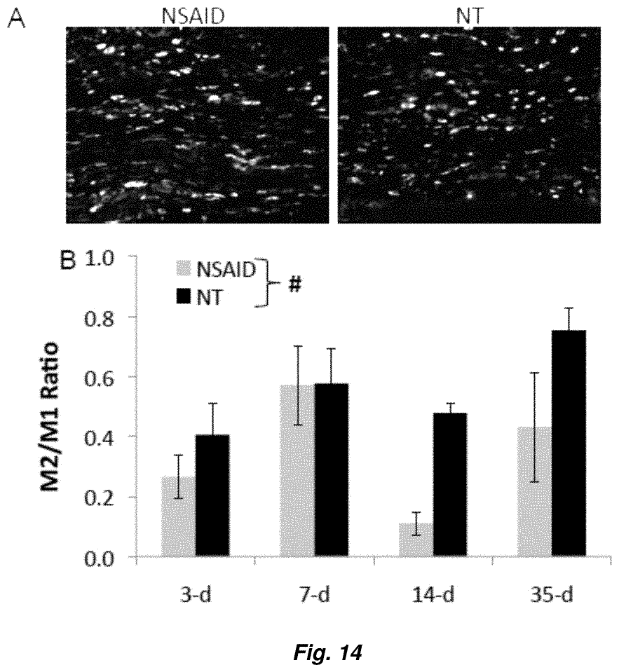

To determine if Aspirin administration altered ECM scaffold mediated macrophage phenotype, CD206+CD68+ (M2-like) and CD86+CD68+ (M1-like) macrophages were immunolabeled and quantified using an automated cell profiler analysis pipeline (FIG. 14). Aspirin treatment altered the phenotype of accumulated macrophages as shown by a reduction in the M2:M1 ratio (main effect for treatment) (FIG. 14). Specifically, Aspirin elicited both a reduction in M2 (CD206+) and an increase in M1 (CD86+) macrophages when compared with non-Aspirin treated animals throughout the experimental time course (FIG. 15). These data suggest that Aspirin alters the typical macrophage phenotype response mediated by ECM scaffolds in vivo both by reducing M2 marker expression and increasing M1 marker expression.

### UBM Mediates COX2 Expression and Prostaglandin Secretion In Vitro.

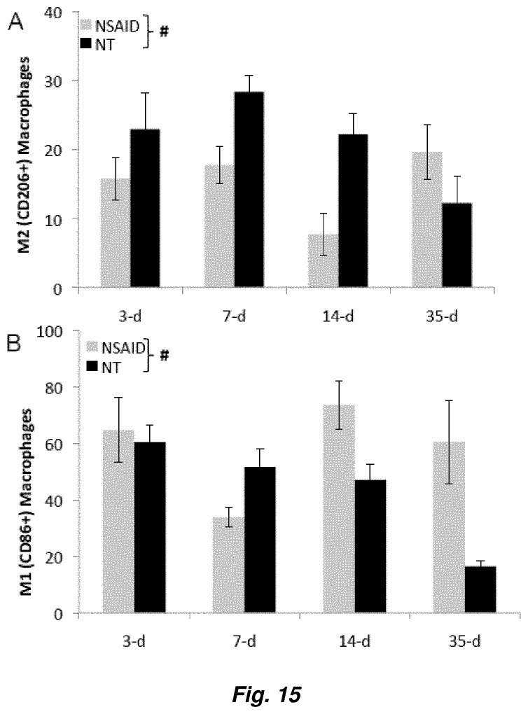

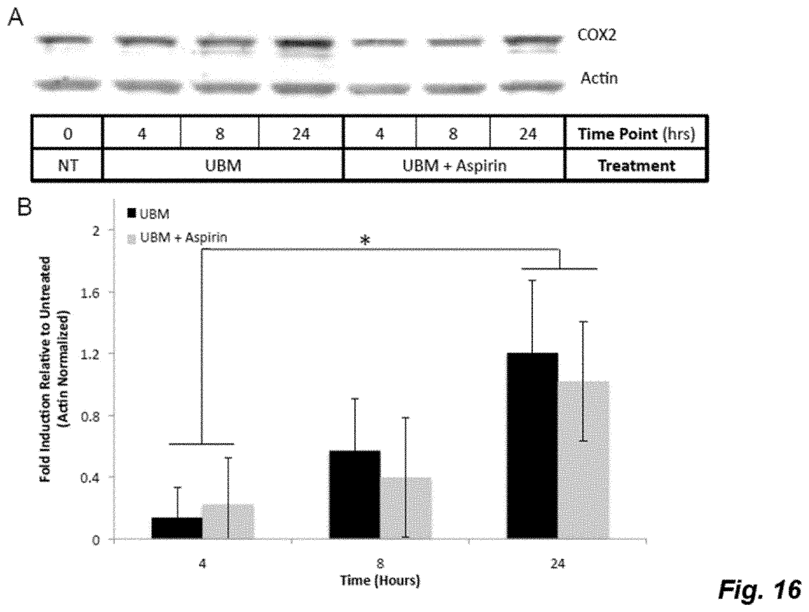

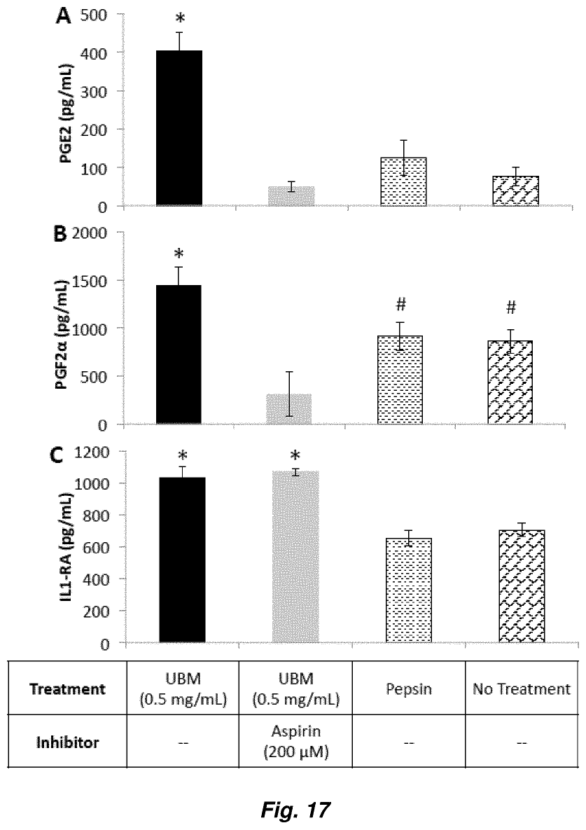

To determine if Aspirin inhibited UBM mediated COX2 expression or secretion of COX1/2 dependent small molecules, THP1 macrophage-like cells were treated with either UBM or UBM and Aspirin for 4, 8, and 24 hours. Western blots of cell lysates (FIG. 16A) showed a steady increase in COX2 expression over the time course reaching a 1.2 fold maximum increase at 24 hours (FIG. 16B). Aspirin did not cause any changes in COX2 expression, consistent with in vivo data. To determine the effect of Aspirin on the downstream products of COX2, the PGE2 and PGF2α concentration in culture supernatants was measured at 48 hours (FIGS. 17A and 17B). Treatment with Aspirin reduced production of both PGE2 and PGF2α down to basal levels. Secretion of a non-COX1/2 dependent factor (IL-1RA) was also examined. Aspirin did not cause any significant drop in IL-1RA secretion suggesting that the dose of Aspirin used was not cytotoxic to the cells (FIG. 17C). The concentration of TNFα and IL-1β in the culture supernatants was also quantified over a 72 hour time course (Supplemental FIG. 18). Minimal concentrations of TNFα or IL-1β were observed over the 72 hour timecourse suggesting that the UBM mediated production of prostaglandins is not merely a side effect of an acute pro-inflammatory response but rather a directed and controlled constructive remodeling response.

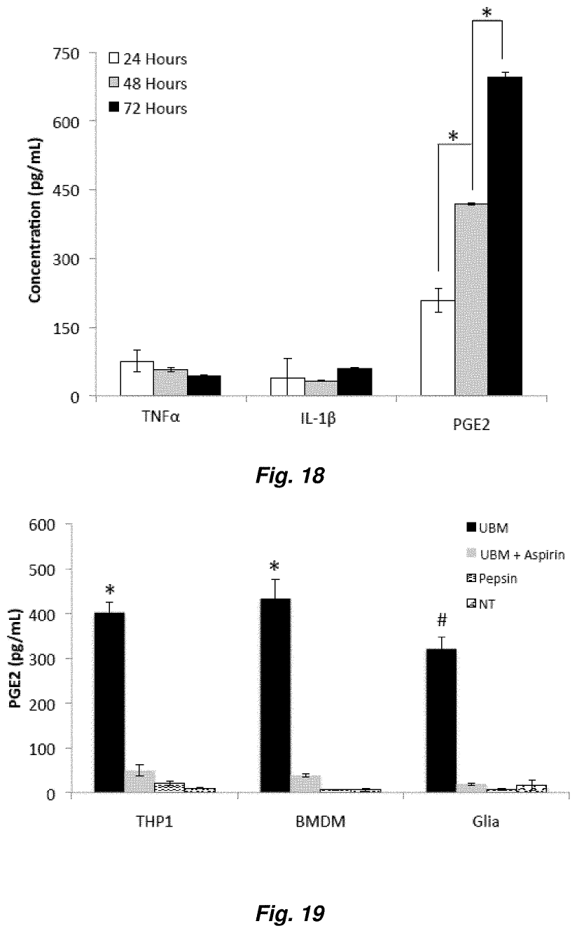

To validate the use of THP1 cells as a model system, PGE2 expression was examined in primary rat bone marrow derived macrophages and brain derived microglia. Treatment of primary cells with UBM for 48 hours mediated similar increases in PGE2 compared to THP1 cells (FIG. 19). Inhibition of COX1/2 with Aspirin reduced PGE2 production to baseline levels in all three cell types. Collectively, these data suggest that UBM activation of PGE2 production is not restricted to the THP1 cell line, and inhibition of COX1/2 is also observed in primary cells.

### The Effect of Aspirin on Macrophage Phenotype.

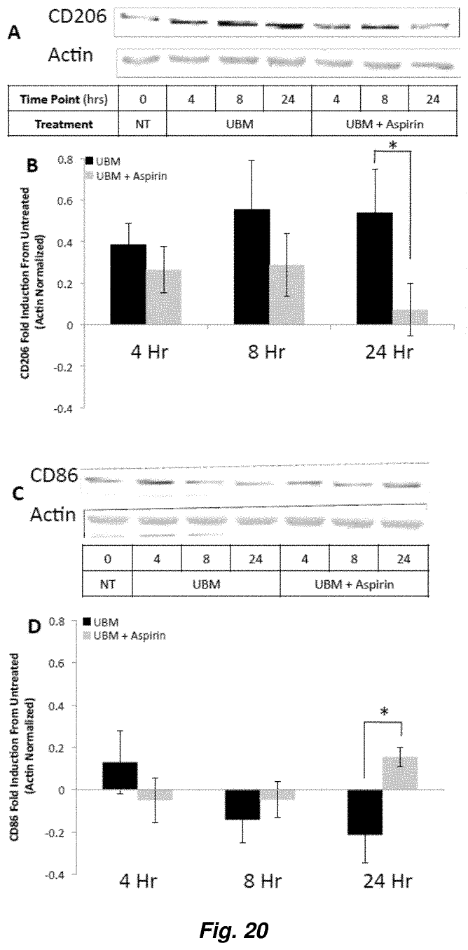

To test the effects of UBM and COX1/2 inhibition on macrophage phenotype, THP1 cells were treated with UBM and changes in their CD206 and CD86 expression profile were determined by western blotting (FIGS. 20A and 20C). Treatment with UBM increased CD206 expression (0.4 fold-0.57 fold above control) at 4, 8, and 24 hours post treatment (FIG. 20B). Increases in CD206 expression were concurrent with a drop in CD86 expression that reached a maximum reduction (0.2 fold) at 24 hours (FIG. 20D). Treatment with aspirin did not alter CD206 expression at the early time points. However, at 24 hours a significant reduction (91%) in CD206 expression was observed with Aspirin treatment. Likewise, treatment with Aspirin caused a steady increase in CD86 expression reaching a maximum 0.15 fold increase at 24 hours that was significantly higher than UBM treatment alone (0.2 fold decrease). Collectively, these data suggest that Aspirin treatment inhibits the UBM initiated M2 bias in macrophages by decreasing CD206 expression and increasing CD86 expression.

### Aspirin Treatment Reduces Myogenesis In Vitro.

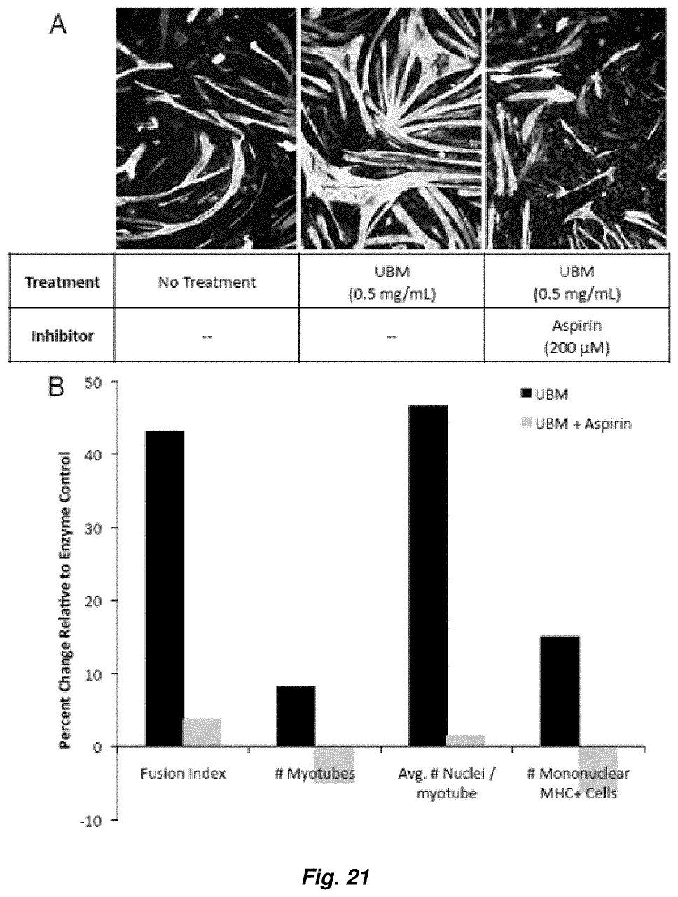

A dynamic interplay between macrophages and skeletal muscle cells is an important component of the skeletal muscle repair/regeneration process following injury. Recently, the secreted products from ECM scaffold induced M2-like macrophages were found to stimulate myogenesis of skeletal muscle progenitor cells in vitro (Sicari B M, Dziki J L, Siu B F, Medberry C J, Dearth C L, Badylak S F. The promotion of a constructive macrophage phenotype by solubilized extracellular matrix. Biomaterials 2014; 35(30):8605-12). To more accurately replicate the kinetics of this interaction between an ECM scaffold, macrophages, and skeletal muscle cells over time; a co-culture system was utilized in which THP1 cells were placed in the same culture well as C2C12 myoblasts, via a transwell insert. UBM (0.5 mg/mL) with or without Aspirin (200 μM) was added to the THP1 cell insert only and the system was incubated for 48 hours. Addition of UBM caused robust increases in several indices of myogenesis, including fusion index (43%), total number of myotubes (8%), average number of nuclei per myotube (47%), and the number of mononuclear MHC+ cells (15%) (FIG. 21). These data indicate that UBM treatment causes macrophages to secrete factors that facilitate myoblast differentiation and fusion into myotubes. Co-administration of aspirin with UBM reduced these myogenesis metrics down to control levels (FIG. 21). Collectively, these data suggest that a COX1/2 inhibitor impairs the ability of macrophages to secrete pro-myogenic factors in response to UBM.

### Discussion

ECM scaffolds facilitate constructive and site appropriate tissue remodeling when implanted into a variety of tissue sites including skeletal muscle. However, occasionally these materials fail to induce or only partially induce a constructive tissue remodeling response with some patients showing robust functional improvement and others showing little improvement along with a marked inflammatory response. One potential source of these divergent results may lie in the post-operative regimen prescribed for these patients (e.g., NSAID administration, physical therapy, etc.). The results presented herein suggest that Aspirin can negatively impact the constructive remodeling events elicited by ECM scaffolds, in terms of bona fide myogenesis and macrophage polarization.

Previous studies utilizing knockout animals or small molecule inhibitors have described a vital role of COX2 activity in endogenous skeletal muscle repair/regeneration. While this detrimental effect of NSAIDs on endogenous skeletal muscle repair is well described, little is known about the effect of NSAIDs on ECM scaffold mediated skeletal muscle repair/constructive remodeling. The current study demonstrated that administration of Aspirin reduced ECM scaffold mediated myogenesis both in vitro and in vivo. Direct study of myotube formation in the THP1/C2C12 co culture system corroborates the in vivo reduction in myogenesis (79%) with a similar reduction in fusion index (92%). Collectively, the data presented herein suggest that COX1/2 activity, namely PGE2 and PGF2α, is a critical component of the ECM scaffold mediated tissue remodeling response.

Macrophages and their acquired phenotype/function, are critical components of the wound healing response. A well-orchestrated phenotypic response beginning with an M1 phenotype during the early (i.e. debridment) part of healing and transitioning to a prolonged M2 phenotype during the repair phase has been well described. A strong M2 bias in the macrophage population is consistently observed when an ECM scaffold is utilized and consistently precedes an improved remodeling outcome. The results presented herein confirm these reports as UBM mediated an increase in M2 marker expression (CD206) and a decrease in M1 marker expression (CD86) both in vitro and in vivo. Inhibition of COX2 reversed these trends driving a stronger M1 macrophage response. These findings support several recent studies which have shown that COX2 and its downstream products are an important component in the development of an M2 phenotype. Knockout of the COX2 gene in rodents prevents macrophages from progressing along the M2 spectrum. Even though the results of the present study relies on two markers to describe macrophage phenotype, a number of studies utilizing ECM scaffolds to treat similar (skeletal muscle) and unrelated (esophageal, TMJ) injury models have consistently shown a bias towards CD206 and away from CD86, validating the use of these markers and the results presented herein.

Rather than a traditional histological/mechanical properties assessment of constructive remodeling, the present study investigated the influence of COX2 in mediating several important events in the complex host response to an ECM scaffold material. The COX1/2 inhibitor, Aspirin, was found to augment the ECM scaffold-mediated constructive remodeling response both in an in vitro co-culture system and an in vivo rat model of skeletal muscle injury. While further studies are needed to more completely characterize this response, the results presented herein provide data to substantiate the possibility that the use of NSAIDs may significantly alter tissue remodeling outcomes in regenerative medicine/tissue engineering applications. Thus, the decision to prescribe NSAIDs to manage the symptomatology of inflammation post-ECM scaffold implantation should be approached with care.

Collectively, we have greatly expanded our understanding of the structural components of UBM. We have pinpointed a potential molecular origin (hyaluronan) and also shown the potential utility of this fraction in vivo.

Although the present invention has been described with references to specific details of certain embodiments thereof, it is not intended that such details should be regarded as limitations upon the scope of the invention except in so far as they are included in the claims.

1. A method of preparing one or more biologically active fractions of ECM useful for modulating chemotaxis and proliferation of stem cells, comprising:
a. partially or completely digesting decellularized ECM material prepared from a tissue with an acid protease;
b. neutralizing the digested ECM material to a pH of 7.0-8.0, 7.2-7.8 or 7.4;
c. gelling the neutralized, digested ECM material at a temperature above its Lower Critical Solution Temperature;
d. centrifuging the gelled ECM material to produce a pellet and a supernatant; and
e. separating the supernatant and the pellet, thereby producing a structural and a soluble fraction of the ECM material.

2. The method of claim 1, wherein the acid protease is pepsin.

3. The method of claim 1, in which the decellularized ECM material prepared from the tissue is not dialyzed prior to the partial or complete digestion with the acid protease.

4. The method of claim 1, further comprising dispersing the pellet into an aqueous solution, thereby preparing a solution of structural components of the ECM.

5. The method of claim 4, wherein the pellet is dispersed in the aqueous solution by homogenization.

6. The method of claim 1, further comprising precipitating remaining structural components from the supernatant.

7. The method of claim 6, wherein the structural components are precipitated from the supernatant by increasing the salt concentration in the supernatant.

8. The method of claim 1, further comprising lyophilizing the supernatant.

9. The method of claim 8 further comprising re-hydrating the lyophilized supernatant.

10. The method of claim 9, the supernatant having a volume before lyophilization, and wherein the lyophilized supernatant is re-hydrated to a volume, less than the volume of the supernatant before lyophilization, optionally the lyophilized supernatant is re-hydrated to a volume <10%, 10%, 20%, 25% or 50% of the volume of the supernatant before lyophilization, thereby producing a concentrated solution of soluble ECM components.

11. The method of claim 1, in which the decellularized ECM material is partially digested.

12. The method of claim 11, in which the decellularized ECM material is digested less completely than a digestion of 1 mg/mL lyophilized, powdered ECM material with 1 mg/mL pepsin in 0.01 M HCl for 48 hours.

13. The method of claim 11, in which the decellularized ECM material is digested less completely than a digestion of 10 mg/mL lyophilized, powdered ECM material with 1 mg/mL pepsin in 0.01 M HCl for 48 hours.

14. The method of claim 11, in which hyaluronic acid in the decellularized ECM material is digested less than 50%, 40%, 30%, 25%, 20% or 10%.

15. The method of claim 1, further comprising absorbing into, adsorbing onto, or otherwise dispersing the biologically active fraction of ECM onto or into a biocompatible substrate.

16. The method of claim 15, in which the biocompatible substrate is a mesh, a non-woven, decellularized tissue, a polymer composition, a polymeric structure, a cell growth scaffold, an implant, an orthopedic implant, and intraocular lens, sutures, intravascular implants, stents, or transplants.

17. A composition prepared by the method of claim 1.

18. A medical implantation device for supporting tissue remodeling, cell growth, migration and/or differentiation, comprising a biocompatible substrate and a composition prepared by the method of claim 1, absorbed in, adsorbed to, or otherwise dispersed on or in the biocompatible substrate.

19. A device for supporting tissue remodeling, cell growth, migration and/or differentiation, comprising a biocompatible substrate, and composition prepared by the method of claim 1, absorbed in, adsorbed to, or otherwise dispersed on or in the biocompatible substrate.

20. A method of modulating an immune response in a patient in need thereof, comprising administering to a patient parenterally or topically the biologically active fraction of ECM prepared by the method of claim 1 in an amount effective to modify an immune response in the patient.

21. The method of claim 20, comprising administering the soluble ECM components from the supernatant to the patient, thereby increasing the macrophage M2 response in the patient.

22. The method of claim 20, comprising administering the structural ECM components from the supernatant to the patient, thereby increasing the macrophage M1 response in the patient.
